(19) United States
(12) Patent Application Publication
(10) Pub. No.: US 2005/0043510 A1
(43) Pub. Date: Feb. 24, 2005

(54) Affinity purification system using troponin molecules as affinity ligands

(76) Inventors: Randolph A. Moeckli, San Carlos, CA (US); Christopher C. Chadwick, West Chester, PA (US)

Correspondence Address: MORRISON & FOERSTER LLP, 755 PAGE MILL RD, PALO ALTO, CA, 94304-1018, US

(21) Appl. No.: 10/820,998

(22) Filed: Apr. 7, 2004

Related U.S. Application Data
(60) Provisional application No. 60/462,483, filed on Apr. 10, 2003.

(51) Int. Cl. C12P 21/04

(52) U.S. Cl. 530/344

(57) ABSTRACT

This invention pertains to the use of a novel set of tags for the immobilization and/or purification of proteins or other biological or organic molecules. The invention provides troponin C, troponin C binding peptide, and troponin I, or active fragments or analogues thereof as convenient tags and affinity ligands for immobilizing, attaching, or purifying proteins or other molecules. Methods for producing troponin-tagged molecules, such as recombinant fusion proteins, are described. Methods for preparing a troponin affinity matrix that is capable of specifically binding its cognate ligand in the presence of calcium, and methods for using such a matrix to purify troponin-tagged molecules are also described.

### CROSS REFERENCE TO RELATED APPLICATIONS

This application claims the benefit of U.S. Provisional Application No. 60/462,483, filed on Apr. 10, 2003, the disclosure of which is incorporated by reference in its entirety herein.

### FIELD OF THE INVENTION

This invention provides methods and reagents for purification of polypeptides or other molecules. In particular, this invention relates to the use of the troponin C and its cognate ligands troponin C binding peptide and troponin I as protein tags and affinity ligands for use in immobilization and/or affinity purification procedures.

### BACKGROUND OF THE INVENTION

A number of approaches have been developed for the isolation and purification of proteins, particularly recombinant proteins, from other components of a biological sample. One general approach exploits the non-specific affinity of a protein for a substrate. Thus, for example, proteins can be separated based upon their molecular charge using ion exchange chromatography. In such an approach, biological samples are applied to a charged chromatographic matrix, and the various proteins bind to the matrix by reversible, electrostatic interactions. The adsorbed proteins are eluted, in order of least to most strongly bound, by increasing the ionic strength or by varying the pH of the elution buffer. However, often the protein of interest will elute with other proteins with similar charge characteristics, rendering this approach undesirable for many applications.

Another general approach utilizes a protein's physical characteristics as a means of purification. For example, a protein may be separated from other proteins based upon its size, using gel filtration or other size separation methods. However, this approach is often inefficient because a protein of interest may co-elute with many other proteins of similar size.

A third general approach, affinity chromatography, is more specific and much more efficient than the above purification methods, because it makes use of the specific affinity of a protein for a purifying reagent such as an antibody or ligand to which it specifically binds. For example, a protein that is an antigen for an antibody may be purified using that antibody as an affinity ligand, or conversely, an antibody may be purified using its specific antigen as an affinity ligand. Typically, an affinity ligand such as an antibody or antigen is covalently bound to a substrate to form an affinity matrix. The matrix is contacted by a solution which includes the binding partner of the affinity ligand (i.e., a protein of interest), allowing formation of a substrate-bound affinity complex. Formation of the affinity complex may occur in a column. Alternatively, affinity complexes may be formed in solution, with the affinity ligand covalently linked to a solid particle such as sepharose or agarose, with the complexes then isolated by centrifugation. To recover the protein of interest, the affinity complex is destabilized, e.g., by exposure to buffers of very high ionic strength or high or low pH. In another example of affinity purification, immunocomplex formation may be exploited to purify an antigen or antibody by immunoprecipitation. Antigen-antibody complexes may be aggregated and precipitated following formation, followed by release of the antigen (i.e., a protein of interest) from the immunocomplex as described above.

Typically, in affinity purification, it is desirable to have a specific, but relatively low affinity interaction between binding partners in order not to impair the function of the purified protein recombinant protein with a harsh elution protocol. In particular, it is often desirable to elute the purified protein without using a denaturing reagent.

A number of binding pairs are known that are suitable for affinity purification. One member of a binding pair may be used to “tag” a protein of interest, with the other member used as an affinity ligand. Such a protein “tag” may be “fused” recombinantly and expressed to produce a fusion protein with the tag attached. The “tagged” fusion protein is then affinity purified by interaction with the binding partner of the tag and the tag is then optionally cleaved to release pure protein.

A variety of fusion tags have been used to date for recombinant proteins. Such tags include peptides that recognize a specific antibody such as the myc tag (see e.g., Munro and Pelham (1986) Cell, 46: 291-300; Ward et al. (1989) Nature, 341: 544-546), the Flag-peptide (see e.g., Hopp et al (1988) BioTechnology, 6: 1204-1210), the KT3 epitope (Martin et al. (1990) Cell, 63: 843-849, Martin et al (1992) Science, 255: 192-194), an α-tubulin epitope (Skinner et al (1991) J. Biol. Chem., 266: 14163-14166), and the T7 gene 10 protein peptide tag (Lutz-Freyermuth et al. (1990) Proc. Natl. Acad. Sci., USA, 87: 6393-6397).

Other tags include poly-histidine tails that bind to nickel-chelating agarose (Skerra et al. (1991) BioTechnology, 9: 273-278; Lilius et al (1991) Eur. J. Biochem., 198, 499-504), and the strep-tag, a peptide tag binding to streptavidin (see, e.g., Schmidt and Skerra, (1993) Protein Engineering, 6: 109-122). Still other tags involve a protein domain that forms a complex with a second (macro) molecule e.g. glutathione-S-transferase (Smith and Johnson (1988) Gene 67: 31-40), bovine pancreatic trypsin inhibitor (BPTI) (Borijin and Nathans (1993) Proc. Natl. Acad. Sci., USA, 90, 337-341), maltose binding protein (MBP) (Bedouelle and Duplay (1988) Eur. J. Biochem. 171 541-549; Maina et al. (1988) Gene 74 365-373), or a polypeptide sequence that can be biotinylated, allowing interaction with avidin or streptavidin (Schatz, P. J. (1993) Bio/Technology 11, 1138-1143).

Currently-available fusion tagging systems suffer from drawbacks that include low solubility of tagged fusion proteins and inability to purify tagged proteins under denaturing or reducing conditions that are common in many protein purification protocols. Thus, there is a need for an improved tagging system that allows tagged fusion proteins to remain soluble and is stable to affinity purification under denaturing and/or reducing conditions.

### SUMMARY OF THE INVENTION

The invention provides methods and compositions for tagging and purification of molecules such as polypeptides, using troponin C and its cognate ligands, such as troponin C binding peptide or troponin I.

In one aspect, the invention provides a method for purifying a polypeptide or other molecule that includes a troponin “tag” (e.g., a molecule of troponin C, troponin C binding peptide, or troponin I, or a fragment, mutated form, concatenation, insertion or deletion mutant, or other modification thereof that is capable of binding to a cognate ligand that is attached to an affinity matrix), including contacting the troponin-tagged molecule with an affinity matrix that comprises a cognate ligand of the troponin tag, thereby immobilizing the troponin-tagged molecule on the affinity matrix. In some embodiments, the troponin tag includes troponin C and the affinity matrix includes a cognate ligand of troponin C, such as a troponin C binding peptide or troponin I. In other embodiments, the troponin tag includes a cognate ligand of troponin C, such as a troponin C binding peptide or troponin I, and the affinity matrix includes troponin C. Troponin-tagged molecules are immobilized on the affinity matrix and released by adding a releasing agent. Typically, binding to the affinity matrix is facilitated by the presence of calcium, and the releasing agent is an agent that chelates calcium, such as, for example, EDTA, EGTA, BAPTA, citrate, or phosphate. Alternatively, the releasing agent may be an excess of a cognate ligand of the troponin tag, or may be a protease. Typically, the affinity of binding between the troponin tag and its cognate ligand in the presence of calcium comprises a K_{d} greater than 10 nM, often about 100 nM to 1 μM, often about 400 to about 600 nM, typically about 450 to about 500 nM, sometimes about 450 nM.

In some embodiments, the troponin-tagged molecule to be purified is a fusion protein that comprises a troponin molecule and a polypeptide that is not a troponin molecule. In some embodiments, the fusion protein is produced recombinantly, optionally expressed from an expression vector which may be comprised within a host cell. In some embodiments, an expression vector may include a polynucleotide that includes a sequence that encodes a troponin molecule (e.g., troponin C, troponin C binding peptide, or troponin I) and a sequence encoding a polypeptide that is not a troponin molecule inserted into a multiple cloning site. A troponin-tagged polypeptide may include a troponin tag at the N-terminus, the C-terminus, and/or at an internal position attached to an amino acid residue within the amino acid sequence between the N-terminus and the C-terminus. Optionally, a linker may be included between the troponin tag and the polypeptide. In some embodiments, the linker is a polypeptide that includes a protease recognition site. In embodiments in which the troponin-tagged polypeptide includes a protease recognition site, the polypeptide may be released from the affinity matrix by protease cleavage at the protease recognition site to cleave the polypeptide from the troponin tag.

In some embodiments, the troponin-tagged molecule includes at least one molecule of troponin C, or a fragment or analogue thereof that is capable of specifically binding to an affinity matrix comprising a cognate ligand of troponin C, such as a troponin C binding peptide or troponin I. In one embodiment, the affinity matrix comprises a troponin C binding peptide. The troponin C binding peptide may comprise the sequence SRLDYLKSSLLHLGSR (SEQ ID NO:1), or a fragment or analogue thereof that is capable of specifically binding troponin C. The troponin C binding peptide may further include a cysteine residue, for example at the N-terminus, permitting the affinity matrix to be formed by reacting the troponin C binding peptide with a thiol reactive matrix. The troponin C binding peptide affinity matrix may include a substrate of cross-linked polysaccharide, agarose, ceramic, metal, glass, plastic, or cellulose. A troponin C-tagged molecule may be released from a troponin C binding peptide affinity matrix with a chelating agent or a protease, as discussed above, or by adding an excess of troponin C binding peptide, or a fragment or analogue thereof that is capable of specifically binding troponin C. In another embodiment, the affinity matrix comprises troponin I, or a fragment or analogue thereof that is capable of specifically binding to troponin C. In some embodiments, the troponin I is mutated to eliminate internal cysteine residues or to replace internal cysteine residues with other amino acids, such as, for example, serine or threonine. In some embodiments, the troponin I comprises the sequence CCCSSSSSSSS (SEQ ID NO:3) at the N-terminus or the sequence SSSSSSSSCCC (SEQ ID NO:4) at the C-terminus, and the affinity matrix is formed by reacting the troponin I with a thiol reactive matrix. The troponin I affinity matrix may include a substrate of cross-linked polysaccharide, agarose, ceramic, metal, glass, plastic, or cellulose. A troponin C-tagged molecule may be released from a troponin I affinity matrix with a chelating agent or a protease, as discussed above, or by adding an excess of troponin I, or a fragment or analogue thereof that is capable of specifically binding troponin C. In some embodiments in which a troponin C-tagged molecule is purified on a troponin I affinity matrix, at least part of the purification may be performed in the presence of a denaturing agent. The denaturing agent may comprise, for example, urea.

In some embodiments, the affinity matrix includes troponin C, or a fragment or analogue thereof that is capable of specifically binding its cognate ligand, e.g., a troponin C binding peptide or troponin I, on a troponin-tagged molecule to be purified. A troponin C affinity matrix may be formed by irreversibly linking troponin C to a substrate via reactive amino groups of amino acid residues of the troponin C molecule. In some embodiments, a troponin C affinity matrix is formed by reacting troponin C with a cyanogen bromide or glyoxal activated matrix. The troponin C affinity matrix may include a substrate of cross-linked polysaccharide, agarose, ceramic, metal, glass, plastic, or cellulose. In some embodiments, a troponin-tagged molecule to be purified on the troponin C affinity matrix includes at least one molecule of a troponin C binding peptide, or a fragment or analogue thereof that is capable of specifically binding to troponin C. The troponin C binding peptide may comprise the sequence SRLDYLKSSLLHLGSR (SEQ ID NO:1), or a fragment or analogue thereof that is capable of specifically binding troponin C. A troponin C binding peptide-tagged molecule may be released from a troponin C affinity matrix with a chelating agent or a protease, or by adding an excess of troponin C, or a fragment or analogue thereof that is capable of specifically binding the troponin C binding peptide. In some embodiments, a troponin-tagged molecule to be purified on the troponin C affinity matrix includes at least one molecule of troponin I, or a fragment or analogue thereof that is capable of specifically binding troponin C. In some embodiments in which a troponin I-tagged molecule is purified on a troponin C affinity matrix, at least part of the purification procedure may be performed in the presence of a denaturing agent. The denaturing agent may comprise, for example, urea. A troponin I-tagged molecule may be released from a troponin C affinity matrix with a chelating agent or a protease, or by adding an excess of troponin C, or a fragment or analogue thereof that is capable of specifically binding troponin C.

In another aspect, the invention provides compositions for purification of troponin-tagged molecules. Compositions include troponin molecules (e.g., troponin C, troponin C binding peptide, and/or troponin I) attached to a substrate to form an affinity matrix. In some embodiments, the substrate of the affinity matrix includes cross-linked polysaccharide or agarose. In some embodiments, the affinity matrix includes a material selected from the group consisting of ceramic, metal, glass, plastic, or cellulose. The troponin molecule may be attached to the affinity substrate via a linker, which may be selected from the group consisting of an alkyl chain, a carbohydrate, and a polypeptide. A troponin molecule may be modified to facilitate attachment to a substrate to form an affinity matrix, for example, by addition of a terminal cysteine residue to allow attachment to a thiol reactive matrix, such as, for example, an iodoacetic acid activated agarose. The invention also provides a troponin C affinity matrix with a bound troponin C binding peptide- or troponin I-tagged molecule, a troponin C binding peptide affinity matrix with a bound troponin C-tagged molecule, and a troponin I affinity matrix with a bound troponin C-tagged molecule.

In another aspect, the invention provides methods for producing troponin C, troponin C binding peptide, and troponin I affinity matrices. In one embodiment, the invention provides a method for producing an affinity matrix comprising troponin C, or a fragment or analogue thereof that is capable of specifically binding to a troponin C binding peptide or troponin I, the method comprising reacting the troponin C with a cyanogen bromide or glyoxal activated matrix. In one embodiment, a method for producing a troponin C affinity matrix comprises irreversibly linking the troponin C to a substrate via reactive amino groups on the troponin C molecule. In another embodiment, the invention provides a method for producing an affinity matrix comprising a troponin C binding peptide attached to a substrate. In one embodiment, the troponin C binding peptide comprises the sequence SRLDYLKSSLLHLGSR (SEQ ID NO:1), or a fragment or analogue thereof that is capable of specifically binding to troponin C, and further comprises a cysteine residue at the N-terminus, and the method comprises reacting the troponin C binding peptide with a thiol reactive matrix. In another embodiment, the invention provides a method for producing an affinity matrix comprising troponin I attached to a substrate, or a fragment or analogue thereof that is capable of specifically binding to troponin C. In one embodiment, the troponin I is mutated to eliminate internal cysteine residues or to replace internal cysteine residues with other amino acids, and the troponin I comprises the sequence CCCSSSSSSSS (SEQ ID NO:3) at the N-terminus or the sequence SSSSSSSSCCC (SEQ ID NO:4) at the C-terminus, and the method comprises reacting the troponin I with a thiol reactive matrix. In one embodiment, the internal cysteine residues are replaced with serine or threonine residues.

In a further aspect, the invention provides molecules purified according to the methods described above. In some embodiments, the purified molecule is a polypeptide. In one embodiment, the polypeptide is a recombinantly-produced troponin-tagged fusion protein.

In a still further aspect, the invention provides kits including reagents for affinity purification of troponin-tagged molecules according to the methods described above and/or reagents for producing troponin-tagged molecules, and instructions for use. In some embodiments, a kit is provided that includes an affinity matrix comprising troponin C and instructions for use in a method of purifying a troponin C binding peptide- or troponin I-tagged molecule. In some embodiments, a kit is provided that includes an affinity matrix comprising a troponin C binding peptide and instructions for use in a method of purifying a troponin C-tagged molecule. In some embodiments, a kit is provided that includes an affinity matrix comprising troponin I and instructions for use in a method of purifying a troponin C-tagged molecule. In one embodiment, a kit is provided that includes an affinity matrix that includes troponin I and instructions for use in a method of purifying a troponin C-tagged molecule in the presence of a denaturing reagent such as, for example, about 8M urea. In another embodiment, a kit is provided that includes an affinity matrix that includes troponin C and instructions for use in a method of purifying a troponin I-tagged molecule in the presence of a denaturing reagent such as, for example, about 8M urea. In some embodiments, a kit is provided that includes components for producing a troponin-tagged molecule, e.g., a troponin C-, troponin C binding peptide-, and/or troponin I-tagged molecule, such as, for example, an expression vector for production of a troponin-tagged polypeptide or reagents for producing a troponin-tagged molecule via chemical synthesis or chemical conjugation.

In another aspect, the invention provides methods for detecting troponin-tagged molecules. In one embodiment, a troponin-tagged molecule is detected by using an antibody that specifically recognizes the troponin tag. In another embodiment, a troponin C-tagged molecule is detected by contacting a troponin C-tagged molecule with lanthanide ions, such as, for example, lanthanum, terbium, europium, or gadolinium ions. In one embodiment, a troponin C-tagged molecule, e.g., a troponin C-tagged fusion protein, is detected by luminescence of lanthanide ions bound to troponin C. The luminescent detection may be used, for example, for high throughput screening, study of receptor-ligand interaction, or study of binding kinetics.

### BRIEF DESCRIPTION OF THE DRAWINGS

FIG. 1 schematically illustrates a troponin affinity tag and capture system of this invention in an embodiment in which a troponin C fusion protein is purified on a troponin C binding peptide affinity matrix.

FIG. 2 illustrates key features of an embodiment of a troponin C expression vector. PrT7 refers to the T7 promoter, T refers to a thrombin recognition site, and MCS refers to a multiple cloning site.

FIG. 3 shows a Coomassie blue stained SDS-PAGE gel of a troponin C (TnC)-Jo1 purification. A 12.5% Laemmli mini-gel was used with a sample load of 5 μL on a troponin C binding peptide (TBP)-agarose column. Lane 1 shows the column load, lane 2 is representative of the column flow-through fractions, lane 3 represents the column wash, and lanes 4-8 represent fractions from the elution after addition of EDTA.

FIG. 4 shows a Coomassie blue stained SDS-PAGE gel of a purification of a TnC-LaΔC (rabbit skeletal muscle troponin C fused to the N-terminus of a C-terminal truncated version of the human La/SSB autoimmune antigen) fusion protein on a TBP-agarose column. A 12.5% Laemmli mini-gel was used with a sample load of 5 μL. Lane 1 shows the column load, lanes 2-5 show column flow-through fractions; lanes 6-8 show column wash fractions, and lanes 9-14 show fractions from the elution after addition of EDTA.

FIG. 5 shows a Coomassie blue stained SDS-PAGE gel that illustrates the purification of a troponin C-tagged full length La/SSB on a TBP-agarose column. A 12.5% Laemmli mini-gel was used with a sample load of 5 μL. Lane 1 shows the column load, lanes 2-5 show column flow-through fractions, lanes 6-8 show column wash fractions, and lanes 9-14 show fractions from the elution after addition of EDTA.

FIG. 6 shows an SDS PAGE gel of TBP-La/SSB (lane 1), TnC-Jo1 (lane 2), TnC-LaΔC (lane 3), and TnC-La/SSB (lane 4), together with a corresponding Western blot probed with an anti-rabbit skeletal muscle troponin C antibody.

FIG. 7 illustrates key features of an embodiment of a TBP expression vector. PrT7 refers to the T7 promoter and MCS refers to the multiple cloning site.

FIG. 8 shows a Coomassie blue stained SDS-PAGE gel that illustrates the purification of a troponin C binding peptide-tagged C-terminal truncated La/SSB fusion protein on a troponin C-sepharose column. A 12.5% Laemmli mini-gel was used with a sample load of 5 μL. Lane 1 shows the column load, lanes 2-5 show column flow-through fractions, lanes 6-8 show column wash fractions, and lanes 9-14 show fractions from the elution after addition of EDTA.

FIG. 9 shows a Coomassie blue stained SDS-PAGE gel that illustrates the purification of troponin C-tagged Ro52 on a troponin I-agarose column in the presence of 8M urea. Lane 1 shows the column load, lanes 2-4 show flow-through fractions, lanes 5-10 show column wash fractions, and lanes 11-15 show fractions of the elution after addition of EDTA.

FIG. 10 shows a comparison between purification of (A) His-tagged La antigen (His-La) and (B) troponin C-tagged C-terminal truncated La antigen (TnC-LaΔC). Lane 1 shows the column load, lane 2 shows column flow-through, and lane 3 shows the peak fraction of eluted fusion protein. Arrows to the right of the figure illustrate breakdown products or incomplete translation products of the TnC-LaΔC fusion protein.

FIG. 11 shows a Coomassie blue stained SDS-PAGE gel that illustrates thrombin protease cleavage of a troponin C-tagged LaΔC molecule (TnC-LaΔC). Lane 1 shows untreated TnC-LaΔC. Lanes 2-5 illustrate thrombin cleavage of the fusion protein with increasing amounts of thrombin. Lane 5 demonstrates complete cleavage of the TnC tag from LaΔC. Lane 1: no thrombin. Lane 2: 0.0125 units thrombin. Lane 3: 0.025 units thrombin. Lane 4: 0.05 units thrombin. Lane 5: 0.1 units thrombin.

### DETAILED DESCRIPTION

This invention pertains to a novel affinity purification system and to methods of use thereof. In particular, this invention includes the use of troponin C and polypeptides that bind to troponin C, such as troponin C binding peptide and troponin I, as fusion tags and affinity reagents.

Troponin C is a very acidic protein (high negative charge at neutral pH), thus often imparting solubility to fusion partner proteins. Further, the interaction of troponin C with a cognate ligand such as troponin C binding peptide or troponin I is stable in the presence of high concentrations of reducing agents, such as 50 mM β-mercaptoethanol. Further, troponin C-troponin I binding is stable in the presence of a high concentration of a denaturant, such as 8M urea. Thus, the troponin system described herein overcomes limitations of previously-described tagging systems and provides very powerful tools for affinity purification.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, for example, Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction (Mullis et al., eds., 1994); Current Protocols in Immunology (J. E. Coligan et al., eds., 1991); The Immunoassay Handbook (David Wild, ed., Stockton Press NY, 1994); and Methods of Immunological Analysis (R. Massayeff, W. H. Albert, and N. A. Staines, eds., Weinheim: VCH Verlags gessellschaft mbH, 1993).

Definitions

The terms “polypeptide,” “peptide,” and “protein” are used interchangeably herein to refer to a polymer of amino acid residues. The terms apply to amino acid polymers in which one or more amino acid residue is an analogue of a corresponding naturally occurring amino acid, as well as to naturally occurring amino acid polymers. The term also includes variants on the traditional peptide linkage joining the amino acids making up the polypeptide.

The terms “nucleic acid” or “oligonucleotide” or “polynucleotide” or grammatical equivalents herein refer to at least two nucleotides covalently linked together. A nucleic acid of the present invention is preferably single-stranded or double-stranded and will generally contain phosphodiester bonds, although in some cases, as outlined below, nucleic acid analogs are included that may have alternate backbones, comprising, for example, phosphoramide (Beaucage et al. (1993) Tetrahedron 49(10):1925 and references therein); Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321), O-methylphosphoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097), non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580), “Carbohydrate Modifications in Antisense Research,” Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp. 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The terms “binding partner” or a “member of a binding pair” or “cognate ligand” refer to molecules that specifically bind other molecules to form a binding complex such as antibody/antigen, lectin/carbohydrate, nucleic acid/nucleic acid, receptor/receptor ligand (e.g. IL-4 receptor and IL-4), avidin/biotin, etc. Cognate ligands of troponin C include troponin C binding peptide and troponin I.

The term “troponin C” refers to full-length skeletal muscle or cardiac muscle troponin C from any vertebrate species such as for example human, rabbit, cow, pig, mouse, rat, or sheep, and fragments, sequence variants, mutated forms, modified forms, and analogues thereof that retain the ability to bind to a cognate ligand such as troponin C binding peptide or troponin I in the presence of calcium ions.

The term “troponin I” refers to fill-length skeletal muscle or cardiac muscle troponin I from any vertebrate species, such as for example human, rabbit, cow, pig, mouse, rat, or sheep, and fragments, sequence variants, modified forms, and analogues thereof that retain the ability to bind to a cognate ligand such as troponin C in the presence of calcium ions, optionally in the presence of a denaturant such as urea.

The term “troponin C binding peptide” refers to a polypeptide having the sequence SRLDYLKSSLLHLGSR (SEQ ID NO:1), and to fragments, sequence variants, modified forms, and analogues thereof that retain the ability to bind to troponin C in the presence of calcium ions. In one embodiment, troponin C binding peptide consists of the sequence SRLDYLKSSLLHLGSR (SEQ ID NO:1). In another embodiment, troponin C binding peptide consists essentially of the sequence SRLDYLKSSLLHLGSR (SEQ ID NO:1). In the presence of calcium, troponin C binding peptide is capable of binding with high affinity to troponin C but not to calmodulin.

In a “chimeric molecule,” two or more molecules that are capable of existing separately are joined together to form a single molecule having the desired functionality of all of its constituent molecules. The constituent molecules of a chimeric molecule can be joined synthetically by chemical conjugation or, where the constituent molecules are all polypeptides, polynucleotides encoding the polypeptides may be fused together recombinantly such that a single continuous polypeptide is expressed. Such a chimeric polypeptide is termed a “fusion protein.” A “fusion protein” is a chimeric molecule in which the constituent molecules are all polypeptides and are attached (fused) to each other such that the chimeric molecule forms a continuous single chain. The various constituents can be directly attached to each other or can be coupled through one or more peptide linkers.

A “spacer” or “linker” as used in reference to a chimeric molecule refers to any molecule that links or joins the constituent molecules of the chimeric molecule. Where the chimeric molecule is a fusion protein, the linker may be a peptide that joins the proteins comprising a fusion protein. Although a spacer generally has no specific biological activity other than to join the proteins or to preserve some minimum distance or other spatial relationship between them, the constituent amino acids of a peptide spacer may be selected to influence some property of the molecule such as the folding, net charge, or hydrophobicity. A peptide linker may optionally include a site for digestion by a protease, for separation of the fused constituent polypeptides.

A “troponin tag” or “tag” as used herein refers to a “troponin molecule” (e.g., a molecule of troponin C, troponin C binding peptide, or troponin I) that is attached to a polypeptide or other biological or organic molecule such as, for example, a nucleic acid, an antibody, a lectin, a sugar, a carbohydrate.

The terms “troponin-tagged” or “tagged” molecule refer to a polypeptide or other biological or organic molecule, including, but not limited to, a nucleic acid, lipid, polysaccharide, carbohydrate, or lectin, to which a troponin molecule is attached to form a chimeric molecule. The attachment may be via chemical conjugation. Alternatively, a polynucleotide encoding the troponin molecule may be fused recombinantly to polynucleotide encoding a polypeptide to form a fusion protein. A polypeptide to which a troponin molecule is attached is referred to as a “troponin-tagged polypeptide.” A “troponin-tagged fusion protein” refers to a fusion protein comprising a fusion between a troponin molecule and another polypeptide that is not a troponin molecule. Preferred tagged moieties can be immobilized and/or purified using the troponin tag in accordance with affinity methods of the invention.

A “target” molecule refers to a molecule to which a troponin molecule is to be attached, typically a polypeptide or other molecule that one desires to immobilize and/or purify.

The terms “capture ligand,” “affinity reagent,” and “affinity ligand” are used interchangeably to refer to an agent that specifically binds a cognate ligand with high affinity. Such agents may be attached to a support, termed “substrate” or “matrix material” herein, to form an “affinity matrix.” An affinity matrix of this invention comprises an affinity reagent that is a troponin molecule, such as a troponin C, troponin C binding peptide, or troponin I molecule, or a derivative or fragment thereof that is capable of binding its respective cognate ligand. A cognate ligand may be “immobilized” or “retained” or “bound” on such a matrix until release with a releasing agent.

A “releasing agent” refers to a composition that is capable of releasing an immobilized, bound molecule from an affinity matrix (e.g. releases a bound troponin-tagged molecule from a troponin affinity matrix). Releasing agents of this invention can work through a variety of mechanisms including sequestering divalent cations, denaturation of a protein, and protease digestion to separate a bound tag from the molecule to which it is attached.

A “denaturing reagent” refers to a reagent that denatures a protein, e.g., urea or detergents.

The terms “isolated,” “purified,” and “biologically pure” refer to material which is substantially or essentially free from components which normally accompany it as found in its native state, such as for example in an intact biological system.

A “recombinant expression cassette” or “expression cassette” is a nucleic acid construct, generated recombinantly or synthetically, with nucleic acid elements that are capable of effecting expression of a gene or cDNA in hosts compatible with such sequences. Expression cassettes typically include promoters and optionally, transcription termination signals. An expression cassette generally includes a nucleic acid to be transcribed (e.g., a nucleic acid encoding a desired polypeptide), and a promoter. Additional factors necessary or helpful in effecting expression may also be used as described herein.

“Reporter genes” are genes or cDNAs that express an easily assayable (detectable and/or quantifiable) product. Detection of the assayable product indicates the expression and/or level of expression of the reporter gene. Reporter genes are well known to those of skill in the art. They include, but are not limited to, genes expressing bacterial chloramphenicol acetyl transferase (CAT), beta-galactosidase (B-gal), green fluorescent protein (GFP) and other fluorescent proteins, various bacterial luciferase genes, e.g., the luciferase genes encoded by Vibrio harveyi, Vibrio fischeri, and Xenorhabdus luminescens, the firefly luciferase gene FFlux, and the like.

The term “kit” refers to a collection of materials, preferably a packaged collection of materials (preferably related materials) to perform a particular function (e.g. to run a screening assay, to express a protein, to culture a cell, to purify a troponin-tagged polypeptide, etc.). A kit may optionally comprise instructional materials describing the use of the materials present in the kit.

Troponin Molecules as Tags or Affinity Ligands

The invention provides troponin-tagged molecules, which may be purified or immobilized using an affinity method of the invention. A troponin-tagged molecule, such as a polypeptide, includes an attached troponin molecule, such as troponin C, troponin C binding peptide, or troponin I.

Troponins are highly characterized molecules well known to those of skill in the art. The molecules vary somewhat, depending on the organism and/or tissue from which they are derived. Nevertheless, the molecules are generally well conserved. Moreover, crystal structures have been determined and various domains well characterized.

Troponin is a globular protein, found in vertebrate skeletal and cardiac muscle, that consists of three subunits, each with a specific function. Troponin C is the calcium binding part of the molecule, while troponin I inhibits the ATPase activity of actomyosin, and troponin T provides the binding of troponin to tropomyosin (Filatov et al. (1999) Biochemistry, 64(9): 969-985). Troponin, through its association with actin and tropomyosin on the thin filament, inhibits the actin-myosin interaction at submicromolar calcium concentrations and stimulates the interaction at micromolar calcium concentrations (Farah and Reinach (1995) FASEB J., 9: 755-767).

There are at least two genes that encode troponin C in the tissues of higher animals. One gene encodes the isoforms characteristic for fast skeletal muscle fibers, whereas the second gene encodes the isoform characteristic for slow skeletal fibers and heart muscle fibers (Filatov et al., supra). The forms of troponin C have low isoelectric points, and they are highly homologous.

Troponin C (Tn-C) forms specific complexes with both troponin I (Tn-I) and troponin T (Tn-T). The crystal structure of troponin C complexed with the N-terminal fragment of Tn-I has been determined (Vassylyev et al (1998) Proc. Natl. Acad. Sci., USA, 95: 4847-4852). In the complex, troponin C has a compact globular shape, in contrast to the elongated dumb-bell shaped molecule of uncomplexed troponin C. The troponin I fragment binds to the N-terminal domain of troponin C in its Ca²⁺-bound conformation. Apparently, the complex formation between the two proteins involves a specific steric configuration and, therefore, the complex has a functional significance.

Troponin I inhibits the Mg²⁺-activated ATPase of actomyosin (identical with the actin and Mg²⁺-activated myosin ATPase). Troponin I is a basic protein that readily complexes with the acidic troponin C, with Ca²⁺ strengthening formation of the complex. Thus, upon muscle stimulation, troponin C binds Ca²⁺ and then complexes with troponin I, which provides a simple mechanism to relieve the inhibition of the actomyosin Mg²⁺-ATPase by troponin I. Several isoforms of troponin I are known in the molecular mass range of 21-24 kDa.

The peptide and nucleic acid sequences of numerous troponins have been identified. Thus, for example, GenBank lists troponin sequences including, but not limited to: Homo sapiens cardiac ventricular troponin (GenBank Accession No: AF020769), Homo sapiens fast skeletal muscle troponin C (GenBank Accession No: M33772), Homo sapiens slow twitch skeletal muscle/cardiac muscle troponin C (GenBank Accession No: M37984), rabbit fast skeletal muscle troponin C (AA900587), chick skeletal muscle troponin C (GenBank Accession No: B1065677), Caenorhabditis elegans troponin C (GenBank Accession No: BG891770), rabbit troponin I from fast skeletal muscle (GenBank Accession No: L04347), Halocynthia roretzi larval troponin I beta (GenBank Accession No: AB001687), Homo sapiens slow skeletal muscle troponin I (GenBank Accession No: J04760), mouse fast fiber troponin I (GenBank Accession No: J04992).

Troponin C binding peptide has been previously described (Hanson Pierce et al. (1998) J Biol Chem, 273(36): 23448-23453). It exhibits 37.5% sequence identity and 56.25% similarity with amino acids 44-69 of the human cardiac troponin I. Calcium dependent binding of the peptide was demonstrated to rabbit skeletal muscle troponin C but not to calmodulin or bovine serum albumin.

Troponin C binds to a cognate ligand, e.g., troponin C binding peptide or troponin I, with high affinity in the presence of calcium, with a K_{d} greater than 10 nM, generally in the range of about 100 nM to about 1 μM, often about 400 to about 600 nM, typically about 450 to about 500 nM, sometimes about 450 nM. K_{d} may be determined by any well known method in the art, including, for example, surface plasmon resonance measurement (see, for example, Pierce et al. (1998) J. Biol. Chem. 273:23448-23453).

In some embodiments, high affinity binding of troponin C to troponin I is stable in the presence of high concentrations of strong protein denaturants, such as for example 8M urea.

A troponin-tagged molecule may be immobilized on an affinity matrix that includes the cognate ligand of the troponin tag. For example a troponin C-tagged polypeptide may be immobilized on a troponin C binding protein affinity matrix in the presence of calcium or another ion that facilitates binding, for example a lanthanide or strontium ion.

Using known nucleic acid and/or amino acid sequences, troponin molecules may be prepared via recombinant expression using standard methods well known to those of skill in the art. Further, troponin-tagged fusion proteins including these sequences may be prepared recombinantly as well.

However, troponin molecules used in this invention are not limited to those that are prepared recombinantly. Particularly, where the molecule is to be used as a chemically conjugated tag or incorporated into an affinity matrix, purified native forms or chemically synthesized molecules are suitable. Methods of purifying troponin C from biological samples are well known to those of skill in the art (e.g., using combinations of DEAE-cellulose chromatography, hydrophobic interaction chromatography and molecular sieve chromatography, see also Al-Hillawi et al. (1994) Eur. J. Biochem. 225: 1195-201). Troponin C binding peptides may be prepared by peptide synthesis, conventional chromatography techniques or by calcium dependent affinity chromatography on a troponin C affinity matrix.

Troponin-tagged polypeptides may include one or more troponin molecules at the C-terminus and/or N-terminus and/or at an internal position on the polypeptide (e.g., attached to amino acid residue side chain(s)). A troponin molecule may be directly attached to a polypeptide or attached via a linker. In some embodiments, the troponin-tagged polypeptide is a recombinantly-produced fusion protein and the linker is a peptide linker. In one embodiment, the peptide linker includes a protease recognition site.

None of the troponin molecules used as tags or affinity ligands in the practice of this invention are limited to native forms. Modified forms of troponins may be used so long as sufficient binding affinity for a cognate ligand is retained. For example, it will be recognized that numerous deletions (e.g., truncations), insertions (e.g., elongations), concatenations, or mutations can be made to troponin C that do not adversely effect and that may even enhance its ability to interact with troponin C binding peptide or troponin I. Such mutations can be made for a number of reasons including, but not limited to, enhancing binding affinity, particularly when the protein is a component of an affinity matrix, improving troponin stability, introduction or modification of residues to facilitate chemical conjugation of the tag, and modification of a nucleic acid or amino acid sequence to improve expression, or to facilitate coupling to a support to form an affinity matrix. Likewise, it will be recognized that numerous deletions, insertions, concatenations, or mutations can be made to troponin C binding peptide or troponin I that do not adversely effect and that may even enhance ability to bind to troponin C and/or may facilitate covalent coupling to a support to form an affinity matrix. For example, as described in Example 1, troponin C binding peptide with the sequence SRLDYLKSSLLHLGSR (SEQ ID NO:1) was modified to the sequence Ac-CGSGSSRLDYLKSSLLHLGSR-amide (SEQ ID NO:2) to facilitate covalent coupling via an N-terminal cysteine residue to iodoacetic acid activated agarose. The resultant affinity matrix was used for the purification of recombinant troponin C fusion polypeptides.

Affinity Purification Methods

The invention provides methods for purifying or immobilizing troponin-tagged molecules. Methods of the invention include contacting the troponin-tagged molecule with an affinity matrix, to which a cognate ligand of the troponin-tagged molecule(s) is attached, in the presence of calcium or another ion that can substitute for calcium, e.g., lanthanide or strontium, to facilitate binding to the cognate ligand of the troponin tag, thereby immobilizing the tagged molecule on the affinity matrix. Binding of a troponin C-tagged molecule to a troponin I matrix or a troponin I-tagged molecule to a troponin C matrix may optionally occur in the presence of a denaturing agent, such as for example, urea. A releasing agent is added to release the immobilized troponin-tagged molecule from the affinity matrix. Generally, using methods of the invention, a substantially pure composition of at least about 80, 85, 90, 95%, 98, or 99% homogeneity, is obtained.

In the presence of calcium (or another ion that can substitute for calcium, for example, a lanthanide or strontium ion), troponin C is capable of specifically binding to a cognate ligand such as troponin C binding peptide or troponin I. These cognate ligands may be used as a tag and a corresponding affinity reagent (capture ligand) to produce an affinity matrix suitable for the immobilization and/or purification of a molecule, such as a protein, bearing a troponin tag. For example, a troponin C-tagged molecule may be purified on a troponin C binding peptide or a troponin I affinity matrix. Alternatively, a troponin C binding peptide-tagged molecule or a troponin I-tagged molecule may be purified on a troponin C affinity matrix. The tagged molecule to be purified is generally contacted with the affinity matrix in a solution or buffer that includes calcium ions (or another ion that can substitute for calcium), to facilitate binding to the matrix. Often, binding of the tagged molecule to the affinity matrix occurs in about 0.1 to about 10 mM, more often about 5 to about 10 mM calcium. The K_{d} for binding of troponin C to troponin C binding peptide or troponin I is greater than 10 nM, generally in the range of about 100 nM to about 1 μM, often about 200 nM to about 800 nM, more often about 300 nM to about 700 nM, even more often about 400 nM to about 500 nM, typically about 450 nM.

In one embodiment, the affinity matrix occupies the interior of a column. A composition that includes a troponin-tagged molecule to be purified may be loaded onto the column in the presence of calcium, the column washed to remove non-binding molecules, and the troponin-tagged molecule is eluted by adding a releasing agent. Optionally, when the cognate ligands are troponin C and troponin I, binding of the troponin-tagged molecule to the affinity matrix may be in the presence of a denaturing agent, for example urea. In one embodiment binding occurs in the presence of about 2 to about 8M, often about 8M urea.

Releasing agents include any composition that is capable of releasing an immobilized troponin-tagged molecule from an affinity matrix that includes a cognate ligand of the troponin tag. In one embodiment, the releasing agent is an agent capable of sequestering or chelating calcium. Suitable calcium chelators are well known to those of skill in the art and include, but are not limited to, EDTA, EGTA, desferal, biphosphonate, 1,2-bis (2-aminophenoxy) ethane-N,N,N′,N′-tetraacetic acid (BAPTA), BAPTA/AM, EGTA/AM, 5N-BAPTA, 5,5′Br₂-BAPTA, fura-2, Quin-2, and the like. In another embodiment, the releasing agent is a protease, for example a serine protease, that separates the molecule to be purified from the troponin tag, which remains bound to the affinity matrix. In embodiments in which the troponin-tagged molecule is a troponin fusion protein, the protein may optionally be engineered to include a protease recognition site between the troponin molecule and the polypeptide of interest. In one embodiment, the protease recognition site is introduced within a peptide linker joining the subject polypeptide and the troponin tag. Nonlimiting examples of protease recognition sites which may be used are the recognition sites for thrombin or factor Xa.

In a further embodiment, the releasing agent is a cognate ligand of the troponin tag. For example, troponin C binding peptide and/or troponin I may be used as a releasing agent for troponin C-tagged molecules bound to a troponin C binding peptide or troponin I affinity matrix. Likewise, troponin C may be used as a releasing agent for troponin C binding peptide- or troponin I-tagged molecules bound to a troponin C affinity matrix.

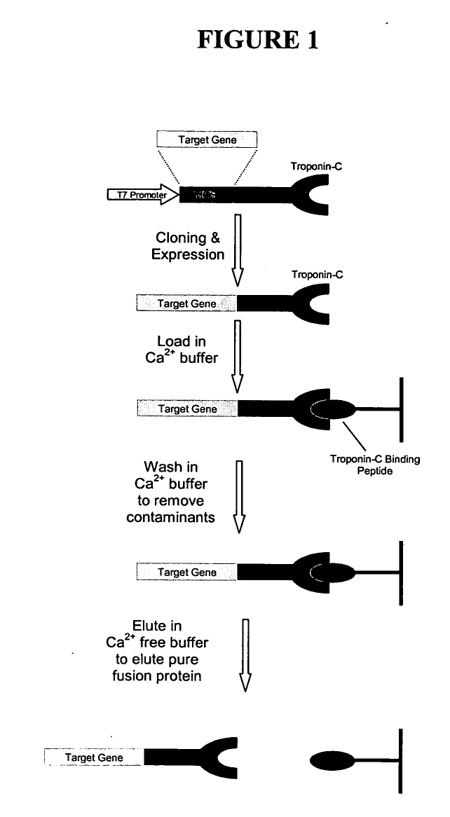

An illustrative embodiment of a troponin affinity tag and capture system is illustrated schematically in FIG. 1. As shown in FIG. 1, in some embodiments, the affinity tag and capture system comprises a vector from which a troponin C-tagged polypeptide is expressed. The vector is designed so that the inserted nucleic acid and the troponin tag nucleic acids are “in frame” and the vector can then express a troponin-tagged polypeptide (e.g., a troponin C polypeptide fusion).

The vector (e.g., plasmid) is transfected into a host cell (e.g., a bacterial cell such as E. coli, an insect cell, a yeast cell, a mammalian cell, etc.) and the fusion protein is expressed (e.g., under control of an inducible or constitutive promoter). After expression, the cells are lysed and the lysate is contacted with an affinity matrix of the invention (e.g., a troponin C binding peptide matrix as illustrated in FIG. 1) in the presence of calcium (Ca²⁺). Under these conditions, the affinity ligand on the affinity matrix binds the troponin tag on the polypeptide immobilizing the polypeptide to the affinity matrix. Contaminants are washed out of the matrix in the presence of calcium. Elution with a chelating agent (e.g., EDTA, EGTA, or the like) then removes the calcium and releases the bound troponin-tagged polypeptide. Alternatively, a recognition site for a protease (e.g., thrombin) may be engineered between the polypeptide to be purified and the troponin tag, and the immobilized polypeptide may be released by protease digestion.

In various embodiments, a troponin tag may be either chemically conjugated to a molecule or expressed as a component of a fusion protein. Methods of preparing “tagged” molecules and of preparing and using affinity matrices according to this invention are described in detail below.

Preparation of a Troponin-Tagged Molecule

A troponin tag can be chemically conjugated to a subject molecule either directly or through a linker. Where the subject molecule is a polypeptide, the troponin tag can be joined to the polypeptide through a peptide linkage to form a fusion protein. Such a fusion protein may be produced either synthetically or recombinantly.

Chemical Conjugation

In some embodiments, a troponin tag is directly conjugated to a polypeptide or other moiety chemically. Means of chemically conjugating molecules are well known to those of skill in the art. Such means will vary according to the structure of the moiety to be tagged, but will be readily ascertainable to those of skill in the art.

Polypeptides typically contain a variety of functional groups, e.g., carboxylic acid (COOH) or free amino (NH₂) groups, that are available for reaction with a suitable functional group on the troponin molecule. A troponin tag may be attached at the N-terminus, the C-terminus, or to a functional group on an interior residue (i.e., a residue at a position intermediate between the N- and C-termini) of a polypeptide. Similarly, nucleic acids and carbohydrates contain functional groups, such as hydroxyl (OH) groups, which are available for reaction. Alternatively, the tag and/or the moiety to be tagged can be derivatized to expose or attach additional reactive functional groups.

In other embodiments, a troponin tag is conjugated to a polypeptide or other biological or organic molecule via a linker. A “linker,” as used herein, is a molecule that is used to join the troponin tag to the moiety that is to be tagged. A number of linker molecules are commercially available, for example from Pierce Chemical Company, Rockford Ill. A preferred linker is capable of forming covalent bonds to both the tag and to the moiety that is to be tagged. Suitable linkers are well known to those of skill in the art and include, but are not limited to, straight or branched-chain carbon linkers, heterocyclic carbon linkers, or peptide linkers. Where the moiety to be tagged is a polypeptide, the linker may be joined to side chain functional groups of constituent amino acids of the tag and/or the polypeptide (e.g., through a disulfide linkage to a cysteine residue). In one embodiment, a linker is joined to alpha-amino and carboxyl groups of the terminal amino acids of the tag and the polypeptide that is to be tagged.

In some embodiments, a bifunctional linker is used that includes one functional group reactive with a pre-existing functionality on the tag, and another group reactive with a pre-existing functionality on the moiety that is to be tagged. In other embodiments, the tag and/or the moiety to be tagged are derivatized. Such derivatization can involve chemical treatment of the moiety to be tagged. For example, glycol cleavage of the sugar moiety of a glycoprotein may be achieved via periodate oxidation to generate free aldehyde groups. The free aldehyde groups on the moiety can then be reacted with free amine or hydrazine groups on a linker to bind the moiety thereto. (See, e.g., U.S. Pat. No. 4,671,958.) Procedures for generation of free sulfhydryl groups on polypeptides are also known. (See, e.g., U.S. Pat. No. 4,659,839.)

In some circumstances, it is desirable to free the tagged molecule from the troponin tag, e.g. after the molecule is isolated on an affinity matrix comprising the cognate ligand. Therefore, conjugates comprising linkages that are cleavable can be used. Cleaving of the linkage to release the tag may be accomplished by enzymatic activity, for example, via protease digestion, or by the use of various chemical reagents. To facilitate cleavage of the tagged molecule from the troponin tag, a cleavable linker may be used. A number of different cleavable linkers are known to those of skill in the art. (See, e.g., U.S. Pat. Nos. 4,618,492; 4,542,225, and 4,625,014.) The mechanisms for release of an agent from these linker groups include, for example, irradiation of a photolabile bond and acid-catalyzed hydrolysis. Alternatively, linkers have been described that may be cleaved by proteolytic enzymes. (See, e.g., U.S. Pat. No. 4,671,958.) In view of the large number of methods that have been reported for attaching a variety of radiodiagnostic compounds, radiotherapeutic compounds, drugs, toxins, and other agents to polypeptides, one skilled in the art will be able to readily determine a suitable method for attaching a troponin tag to a polypeptide or other biological or organic molecule from methods described in the scientific literature.

Preparation of a Troponin-Tagged Fusion Protein

A troponin tag may be attached to a polypeptide via a peptide linkage to form a fusion protein. The troponin tag can be attached to either the amino or carboxyl terminus of the protein. In certain embodiments, the tag is directly attached to the N- or C-terminus of the polypeptide. In other embodiments, the tag is attached to the N- or C-terminus of the polypeptide through a peptide linker.

In embodiments in which a peptide linker is used, the length of the peptide linker sequence may vary without significantly affecting the cognate ligand binding ability of the troponin tag. In some embodiments, a linker of any of 2, 3, 4, 5, 6, 7, 8, 9, 10, 11, 12, 13, 14, or 15 amino acids or amino acid analogues is used. Often, a linker of about 1 to about 50, more often about 1 to about 20, even more often about 2 to about 15, most often about 2 to about 10 amino acids or amino acid analogues is used.

Generally, a linker will have no specific biological activity other than to join the proteins or to preserve some minimum distance or other spatial relationship between them. However, the constituent amino acids of the spacer may be selected to influence some property of the molecule such as the folding, net charge, or hydrophobicity, or in certain embodiments, to introduce a protease recognition site or other cleavage site to facilitate subsequent separation of the troponin tag from the polypeptide. As described in greater detail below, troponin-tagged fusion proteins of the invention can be conveniently produced using either chemical synthesis or recombinant expression methodologies.

1) De Novo Chemical Synthesis.

Troponin-tagged fusion proteins can be synthesized using standard chemical peptide synthesis techniques that are well known to those of skill in the art. In embodiments in which the amino acid sequences are relatively short, the molecule can be synthesized as a single contiguous polypeptide. Where larger molecules are desired, subsequences can be synthesized separately (in one or more units) and then fused by condensation of the amino terminus of one molecule with the carboxyl terminus of the other molecule thereby forming a peptide bond.

Solid phase synthesis in which the C-terminal amino acid of the sequence is attached to an insoluble support followed by sequential addition of the remaining amino acids in the sequence may be used for the chemical synthesis of the polypeptides of this invention. Techniques for solid phase synthesis are described, for example, by Barany and Merrifield, Solid-Phase Peptide Synthesis; pp. 3-284 in The Peptides: Analysis, Synthesis, Biology. Vol. 2: Special Methods in Peptide Synthesis, Part A., Merrifield, et al. (1963) J. Am. Chem. Soc., 85: 2149-2156, and Stewart et al. (1984) Solid Phase Peptide Synthesis, 2nd ed. Pierce Chem. Co., Rockford, Ill.

2) Recombinant Expression.

In some embodiments, troponin-tagged fusion proteins are synthesized using recombinant expression systems. Generally this involves creating a nucleic acid (e.g., DNA) sequence that encodes a polypeptide(s) to be tagged, placing the DNA in an expression cassette in frame with a nucleic acid sequence encoding the troponin tag and under the control of a promoter, and expressing the protein in a host cell.

Using the information provided herein and/or publicly available sequence information for troponin molecules, the troponin C binding peptide, troponin C, or troponin I nucleic acid(s) may be cloned, or amplified by in vitro methods, such as, for example, the polymerase chain reaction (PCR), the ligase chain reaction (LCR), the transcription-based amplification system (TAS), the self-sustained sequence replication system (SSR). A wide variety of cloning and in vitro amplification methodologies are well-known to persons of skill. Examples of techniques sufficient to direct persons of skill through in vitro amplification methods are found in Mullis et al., (1987) U.S. Pat. No. 4,683,202; PCR Protocols A Guide to Methods and Applications (Innis et al. eds) Academic Press Inc. San Diego, Calif. (1990) (Innis); Arrheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science, 241: 1077-1080; Van Brunt (1990) Biotechnology, 8: 291-294; Wu and Wallace, (1989) Gene, 4: 560; and Barringer et al. (1990) Gene, 89: 117.

In one embodiment, the nucleic acids of this invention can be cloned using DNA amplification methods such as polymerase chain reaction (PCR). Thus, for example, the nucleic acid sequence or subsequence is PCR amplified, using a sense primer containing one restriction site (e.g., NdeI) and an antisense primer containing another restriction site (e.g., HindIII). This will produce a nucleic acid encoding the desired sequence or subsequence and having terminal restriction sites. This nucleic acid can then be easily ligated into a vector containing a nucleic acid having the appropriate corresponding restriction sites. Suitable PCR primers can be determined by one of skill in the art using the sequence information. Appropriate restriction sites can also be added to the nucleic acid encoding the desired protein or protein subsequence by site-directed mutagenesis. For example, in one embodiment, a plasmid with sequences encoding a polypeptide to be tagged is cleaved with the appropriate restriction endonuclease(s) and then ligated into an expression vector encoding a troponin molecule, according to standard methods.

In addition, DNA encoding desired fusion protein sequences may be prepared synthetically using methods that are well known to those of skill in the art, including, for example, direct chemical synthesis by methods such as the phosphotriester method of Narang et al. (1979) Meth. Enzymol. 68: 90-99, the phosphodiester method of Brown et al. (1979) Meth. Enzymol. 68: 109-151, the diethylphosphoramidite method of Beaucage et al. (1981) Tetra. Lett., 22: 1859-1862, or the solid support method of U.S. Pat. No. 4,458,066.

Chemical synthesis produces a single-stranded oligonucleotide. This may be converted into double stranded DNA by hybridization with a complementary sequence, or by polymerization with a DNA polymerase using the single strand as a template. One of skill would recognize that while chemical synthesis of DNA is generally limited to sequences of about 100 bases, longer sequences can be obtained by the ligation of shorter sequences. Alternatively, subsequences may be cloned, and cleaved using appropriate restriction enzymes. The fragments may then be ligated together to produce the desired nucleic acid sequence.

A nucleic acid of the invention encoding a polypeptide fused to a troponin tag, e.g., as described above, can be incorporated into a recombinant expression vector in a form suitable for expression in a host cell. As used herein, an “expression vector” is a nucleic acid which, when introduced into an appropriate host cell, can be transcribed and translated into a polypeptide. The terminology “in a form suitable for expression of the fusion protein in a host cell” is intended to mean that the recombinant expression vector includes one or more regulatory sequences operably linked to the nucleic acid encoding the enzyme(s) in a manner that allows for transcription of the nucleic acid into mRNA and translation of the mRNA into the subject protein(s). The term “regulatory sequence” is art-recognized and intended to include promoters, and/or enhancers and/or other expression control elements (e.g., polyadenylation signals). Such regulatory sequences are known to those skilled in the art (see, e.g., Goeddel (1990) Gene Expression Technology: Meth. Enzymol. 185, Academic Press, San Diego, Calif.; Berger and Kimmel, Guide to Molecular Cloning Techniques, Methods in Enzymology 152 Academic Press, Inc., San Diego, Calif.; Sambrook et al. (1989) Molecular Cloning—A Laboratory Manual (2nd ed.) Vol. 1-3, Cold Spring Harbor Laboratory, Cold Spring Harbor Press, NY, etc.).

In certain embodiments, expression vectors are designed specifically for expressing a protein tagged with a troponin molecule. Such expression vectors typically include a nucleic acid sequence encoding a troponin C, troponin C binding peptide, or troponin I molecule and generally also include one or more cloning sites having one or more restriction sites for insertion of a nucleic acid encoding the polypeptide that is to be tagged with the troponin. A cloning site that includes more than one restriction site is termed a “multiple cloning site.” Typically, the cloning site is placed in the vector such that when a nucleic acid encoding a polypeptide is inserted in the cloning site the polypeptide and the troponin tag are in frame and expressed as a fusion protein. As indicated above, the troponin tag can be directly joined to the amino or carboxyl terminus of the subject polypeptide, or joined to the polypeptide through a peptide linker sequence.

The design of a troponin fusion protein expression vector can depend on such factors as the choice of the host cell to be transfected and/or particular polypeptide(s) to be expressed. Generally, the vector is constructed such that a nucleic acid encoding a troponin tag is 5′ or 3′ to a multiple cloning site such that when sequences encoding a polypeptide of interest are inserted into the cloning site, the polypeptide and the troponin tag are in frame and expressed as a fusion protein.

It will be appreciated that desired polypeptides can be operably linked to constitutive promoters for high level, continuous expression. Alternatively, inducible and/or tissue-specific promoters can be utilized. When used in mammalian cells, a recombinant expression vector's control functions are often provided by a promoter, often of viral origin. Promoters include, but are not limited to CMV immediate early, HSV thymidine kinase, early and late SV40, LTRs from retrovirus, and mouse metallothionein-I. Use of appropriate regulatory elements can allow for high level expression of the polypeptide(s) in a variety of host cells.

In some embodiments, a recombinant expression vector for production of a troponin-tagged fusion protein is a plasmid or cosmid. In other embodiments, the expression vector is a virus, or portion thereof, that allows for expression of a nucleic acid introduced into the viral nucleic acid. For example, replication defective retroviruses, adenoviruses and adeno-associated viruses can be used.

Expression vectors may be derived from bacteriophage, including all DNA and RNA phage (e.g., cosmids), or viral vectors derived from all eukaryotic viruses, such as baculoviruses and retroviruses, adenoviruses and adeno-associated viruses, Herpes viruses, Vaccinia viruses and all single-stranded, double-stranded, and partially double-stranded DNA viruses, all positive and negative stranded RNA viruses, and replication defective retroviruses. Another example of an expression vector is a yeast artificial chromosome (YAC), which contains both a centromere and two telomeres, allowing YACs to replicate as small linear chromosomes. A number of suitable expression systems are commercially available and can be modified to produce the vectors of this invention. Illustrative expression systems include, but are not limited to baculovirus expression vectors (see, e.g., O'Reilly et al. (1992) Baculovirus Expression Vectors: A Laboratory Manual, Stockton Press) for expression in insect (e.g. SF9) cells, a wide variety of expression vectors for mammalian cells (see, e.g., pCMV-Script® Vector, pCMV-Tag1, from Stratagene), vectors for yeast (see, e.g., pYepSec1, Baldari et al. (1987) EMBO J. 6: 229-234, pMFa (Kurjan and Herskowitz, (1982) Cell 30: 933-943), pJRY88 (Schultz et al., (1987) Gene 54:113-123), pYES2 (Invitrogen Corporation, San Diego, Calif.), and the like), prokaryotic vectors (see, e.g., arabinose-regulated promoter (Invitrogen pBAD Vector), T7 Expression Systems (Novagen, Promega, Stratagene), Trc/Tac Promoter Systems (Clontech, Invitrogen, Kodak, Life Technologies, MDI Fermentas, New England BioLabs, Pharmacia Biotech, Promega), PL Promoters (Invitrogen pLEX and pTrxFus Vectors), Lambda PR Promoter (Pharmacia pRIT2T Vector), Phage T5 Promoter (QIAGEN), tetA Promoter (Biometra pASK75 Vector), and the like.

The troponin-tagged fusion proteins of this invention can be expressed in a host cell. As used herein, the term “host cell” is intended to include any cell or cell line into which a recombinant expression vector for production of a troponin-tagged fusion protein, as described above, may be transfected for expression of a troponin-tagged fusion protein. Host cells include progeny of a single host cell, and the progeny may not necessarily be completely identical (in morphology or in total genomic DNA complement) to the original parent cell due to natural, accidental, or deliberate mutation. A host cell includes cells transfected or transformed in vivo with an expression vector as described above.

Suitable host cells include, but are not limited to, to algal cells, bacterial cells (e.g. E. coli), yeast cells (e.g., S. cerevisiae, S. pombe, P. pastoris, K. lactis, H. polymorpha, see, e.g., Fleer (1992) Curr. Opin. Biotech. 3(5): 486-496), fungal cells, plant cells (e.g. Arabidopsis), invertebrate cells (e.g. insect cells such as SF9 cells, and the like), and vertebrate cells including mammalian cells. Non-limiting examples of mammalian cell lines which can be used include CHO cells (Urlaub and Chasin (1980) Proc. Natl. Acad. Sci. USA 77: 4216-4220), 293 cells (Graham et al. (1977) J. Gen. Virol. 36: 59), or myeloma cells like (e.g., SP2 or NSO, see Galfre and Milstein (1981) Meth. Enzymol. 73(B):3-46). In one embodiment, the expression system includes a baculovirus vector expressed in an insect host cell.

An expression vector encoding a troponin-tagged fusion protein can be transfected into a host cell using standard techniques. “Transfection” or “transformation” refers to the insertion of an exogenous polynucleotide into a host cell. The exogenous polynucleotide may be maintained as a non-integrated vector, for example, a plasmid, or alternatively, may be integrated into the host cell genome. The term “transfecting” or “transfection” is intended to encompass all conventional techniques for introducing nucleic acid into host cells. Examples of transfection techniques include, but are not limited to, calcium phosphate precipitation, DEAE-dextran-mediated transfection, lipofection, electroporation and microinjection. Suitable methods for transfecting host cells can be found in Sambrook et al. (1989) Molecular Cloning: A Laboratory Manual, 2nd Edition, Cold Spring Harbor Laboratory press, and other laboratory textbooks. Nucleic acid can also be transferred into cells via a delivery mechanism suitable for introduction of nucleic acid into cells in vivo, such as via a retroviral vector (see e.g., Ferry et al. (1991) Proc. Natl. Acad. Sci., USA, 88: 8377-8381; and Kay et al. (1992) Human Gene Therapy 3: 641-647), an adenoviral vector (see, e.g., Rosenfeld (1992) Cell 68: 143-155; and Herz and Gerard (1993) Proc. Natl. Acad. Sci., USA, 90:2812-2816), receptor-mediated DNA uptake (see e.g., Wu, and Wu (1988) J. Biol. Chem. 263: 14621; Wilson et al. (1992) J. Biol. Chem. 267: 963-967; and U.S. Pat. No. 5,166,320), direct injection of DNA (see, e.g., Acsadi et al. (1991) Nature 332: 815-818; and Wolff et al. (1990) Science 247:1465-1468) or particle bombardment (biolistics) (see e.g., Cheng et al. (1993) Proc. Natl. Acad. Sci., USA, 90:4455-4459; and Zelenin et al. (1993) FEBS Letts. 315: 29-32).

Certain vectors integrate into host cells at a low frequency. In order to identify these integrants, in some embodiments a gene that contains a selectable marker (e.g., drug resistance) is introduced into the host cells along with the nucleic acid of interest. Examples of selectable markers include those which confer resistance to certain drugs, such as G418 and hygromycin. Selectable markers can be introduced on a separate vector from the nucleic acid of interest or on the same vector. Transfected host cells can then be identified by selecting for cells using the selectable marker. For example, if the selectable marker encodes a gene conferring neomycin resistance, host cells which have taken up nucleic acid can be identified by their growth in the presence of G418. Cells that have incorporated the selectable marker gene will survive, while the other cells die.

Once expressed, the troponin-tagged proteins can be purified according to standard procedures of the art, including, but not limited to affinity purification, ammonium sulfate precipitation, ion exchange chromatography, or gel electrophoresis (see generally, R. Scopes, (1982) Protein Purification, Springer-Verlag, N.Y.; Deutscher (1990) Methods in Enzymology Vol. 182: Guide to Protein Purification., Academic Press, Inc. N.Y.). In one embodiment, the troponin-tagged fusion proteins are purified on a troponin affinity matrix using the methods described above.

It will be apparent to one of skill in the art that modifications may be made to a troponin-tagged polypeptide without diminishing its biological activity. Some modifications may be made to facilitate the cloning and/or expression of the subject molecule(s). Such modifications are well known to those of skill in the art and include, for example, a methionine added at the amino terminus to provide an initiation site, or additional amino acids introduced as a linker to provide a protease cleavage site, etc.

Preparation of an Affinity Matrix

An affinity matrix is provided for the purification of troponin-tagged moieties. The term “affinity matrix” refers to a substrate to which an affinity reagent (capture ligand) is attached. An affinity matrix of this invention comprises an affinity ligand that is capable of binding to a troponin molecule as described herein, attached to or complexed with a “matrix material” (i.e., a substrate or support). Generally, the affinity matrix comprises a matrix material attached to molecules of troponin C and/or troponin C binding peptide and/or troponin I, or fragments or analogues thereof. Each of these molecules acts as a capture ligand in the presence of calcium (Ca²⁺), or another ion which facilitates binding, to specifically bind its cognate ligand (i.e., troponin C is a cognate ligand for troponin C binding peptide or troponin I, and vice versa). Release of the bound molecule is generally effected by sequestering of the Ca²⁺ ions with a chelating agent, by cleavage (e.g., protease digestion) to effect separation from the bound troponin tag, or by adding an excess of free cognate ligand of the troponin tag.

The capture ligand is covalently attached to or associated with a matrix material. Non-limiting examples of matrix materials include solids, gels, pastes, membranes, slurries, or liquids. Suitable matrix materials include, but are not limited to, glass beads, controlled pore glass, magnetic beads, various membranes or rigid various polymeric resins such as polystyrene, polystyrene/latex, and other organic and inorganic polymers, both natural and synthetic. Illustrative polymers include polyethylene, polypropylene, poly(4-methylbutene), polystyrene, polymethacrylate, poly(ethylene terephthalate), rayon, nylon, poly(vinyl butyrate), polyvinylidene difluoride (PVDF), silicones, polyformaldehyde, cellulose, cellulose acetate, and nitrocellulose. Other materials that can be employed, include paper, glass, minerals (e.g. quartz), ceramics, metals, metalloids, plastics, cellulose, semiconductive materials, or cements. In addition, substances that form gels, such as proteins (e.g., gelatins), lipopolysaccharides, silicates, agarose, and polyacrylamides can be used. Polymers that form several aqueous phases, including, but not limited to, dextrans, polyalkylene glycols or surfactants, such as phospholipids, or long chain (12-24 carbon atoms) alkyl ammonium salts are also suitable.

The matrix material can take any of a number of morphologies. These include, but are not limited to solid or porous beads or other particles, solid surfaces (e.g. array substrates), columns, capillaries, or wells. In some embodiments, a plurality of different materials can be employed to form the affinity matrix, e.g., as laminates, to obtain various properties. For example, protein coatings, such as gelatin can be used to avoid nonspecific binding, simplify covalent conjugation, and/or enhance signal detection.

If covalent binding between the troponin molecule and the matrix material is desired, the surface can be polyfunctional or can be capable of being polyfunctionalized. Functional groups that can be present on the surface and used for linking include, but are not limited to, carboxylic acids, aldehydes, amino groups, cyano groups, ethylenic groups, hydroxyl groups, mercapto groups, and haloacetyl groups (i.e., iodoacetyl groups).

Preferred matrix materials include resins, such as for example synthetic resins (e.g. cross-linked polystyrene, divinyl benzene, etc.), and cross-linked polysaccharides (e.g. cellulose, dextran (sephadex), agarose (sepharose), and the like). In some embodiments, the matrix material includes reactive groups capable of forming a covalent link with a troponin molecule. In one embodiment, the matrix material includes a glyoxal activated agarose. In another embodiment, the matrix material includes a sulfhydryl reactive group. In a still further embodiment, the matrix material is activated with cyanogen bromide. In other embodiments, a troponin molecule is attached to an agarose resin by the use of a cross-linking reagent. Such reagents are well known to those of skill in the art and include, but are not limited to carbodiimides, maleimides, succinimides, and reactive disulfides. In other embodiments, a troponin molecule is joined to a matrix material via a linker. Suitable linkers include, but are not limited to straight or branched-chain carbon linkers, heterocyclic carbon linkers, peptide linkers, and carbohydrates, e.g., as described above.

In one embodiment in which troponin I is used to prepare an affinity matrix, internal cysteine residues are mutated to serines, and an N-terminal peptide linker of the sequence N′-CCCSSSSSSSS-C′ (SEQ ID NO:3) is added to facilitate attachment to Sulfolink® agarose. Interaction of the immobilized Troponin I with a troponin C-tagged fusion protein is typically stable in the presence of 8M urea (see Example 6).

An affinity matrix of the invention can take any convenient form. In certain embodiments, the affinity matrix is packed into a column, a mini-column, or a capillary or microcapillary (e.g., in a “lab on a chip” application), or a capillary electrophoresis tube. In some embodiments, the affinity matrix is suspended in one phase of a multiphase solution. In such embodiments, the affinity matrix thus acts to partition the tagged molecule into that particular phase of the multiphase (e.g., two-phase) system. Such multi-phase purification systems are well suited to large volume/high throughput applications.

In some embodiments, the affinity matrix comprises the walls of a vessel or the walls of a well (e.g., in a microtiter plate). In other embodiments, the affinity matrix comprises one or more porous or non-porous membranes or various gels or hydrogels. In one embodiment, the affinity matrix takes the form of a gel, such as for example a slab gel or a tube gel.

Compositions

The invention provides compositions such as troponin-tagged molecules, affinity matrices that comprise troponin molecules, and molecules purified according to the methods of the invention.

In one aspect, a composition is provided that includes an affinity matrix with an attached troponin molecule (e.g., troponin C, troponin C binding peptide, and/or troponin I), the preparation of which is described above. In some embodiments, the affinity matrix includes a troponin C binding peptide, such as a peptide having the sequence SRLDYLKSSLLHLGSR (SEQ ID NO:1) or a troponin C binding fragment or analogue thereof, attached to a substrate. The substrate may include any of the materials described above. In one embodiment, the substrate includes an agarose. For example, a troponin C binding peptide with either an N- or C-terminal cysteine residue may be linked to agarose via a thioether bond. In other embodiments, the affinity matrix includes troponin I attached to a substrate, preferably via a N- or C-terminal linker.

In another aspect, a composition is provided that includes a molecule purified according to any of the methods described herein. In some embodiments, the troponin-tagged molecule comprises a polypeptide. In one embodiment, the polypeptide is produced recombinantly as a troponin-tagged fusion protein.

Compositions are also provided that comprise any of the troponin containing affinity matrices described herein with one or more noncovalently bound troponin-tagged molecules, generally in the presence of calcium to facilitate binding between a troponin tag and a cognate ligand attached to the affinity matrix.

Kits

The reagents described herein can be packaged in kit form. In one aspect, the present invention provides a kit that includes reagents useful for preparation of troponin-tagged molecules and/or purification of such molecules on a troponin affinity matrix, in suitable packaging. Kits of the invention include any of the following, separately or in combination: a troponin-linked affinity matrix (such as, for example, a cross-linked polysaccharide affinity matrix material (e.g. acrylamide, agarose, etc.) comprising a troponin C, troponin C binding peptide, or troponin I molecule or fragment or analogue thereof), reagents for production of a troponin affinity matrix, reagents for production of a troponin-tagged molecule, such as for example an expression vector for production of a troponin-tagged fusion protein (optionally comprising a multiple cloning site for introduction of a nucleic acid encoding a polypeptide “in frame” with troponin sequences), linkers, buffers, or other reagents for affinity purification of a troponin-tagged molecule according to the methods described above.

Each reagent is supplied in a solid form or liquid buffer that is suitable for inventory storage, and later for exchange or addition into a reaction or culture medium. Suitable packaging is provided. As used herein, “packaging” refers to a solid matrix or material customarily used in a system and capable of holding within fixed limits one or more of the reagent components for use in a method of the present invention. Such materials include glass and plastic (e.g., polyethylene, polypropylene, and polycarbonate) bottles, vials, paper, plastic, and plastic-foil laminated envelopes and the like. The kits can optionally further comprise a calcium chelator (e.g. EDTA, EGTA, or the like), a column or other suitable structure for containing an affinity matrix, cells for expressing a polypeptide, transfection reagent(s), and/or other reagents for use in the methods of the invention as described above.

In addition, the kits optionally include labeling and/or instructional materials providing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to Internet sites that provide such instructional materials.

Purification of Troponin-Tagged Molecules Via Ion Exchange

Due to the acidic nature of troponin C, a troponin C-tagged molecule, such as for example a troponin C-tagged polypeptide, may be purified using a ion exchange matrix such as for example DEAE (e.g., DEAE cellulose, acrylamide, or sepharose, or a cross-linked DEAE polysaccharide). The troponin C-tagged molecule can optionally be purified on the ion exchange matrix in the presence of a denaturing reagent, such as urea. The bound moiety can be eluted by increasing the ionic strength and/or decreasing the pH of the eluant. In one embodiment, a troponin C-tagged molecule is purified and released in the presence of a denaturant, for example, about 8M urea, on DEAE cellulose with a gradient of increasing sodium chloride concentration.

Binding of Lanthanide Ions by Troponin C

Troponin C is known to bind lanthanide metal ions. (Wang CL. et al., (1981) Biochemistry 28:2439-44) “Lanthanides” include, e.g., lanthanum, terbium, europium, gadolinium, and other metals in the lanthanide series of the periodic table of the elements. In one embodiment, a troponin C-tagged molecule, prepared and/or purified according to the methods described above, is contacted with a solution that contains lanthanide ions, which luminesce after binding to troponin C, following excitation at 280 nm via adjacent troponin C aromatic amino acid residues. Such a molecule comprising lanthanide may be used, for example in high throughput screening studies, receptor-ligand interaction or kinetic studies, etc., by exploiting the capability of the lanthanide to luminesce. In one embodiment, a troponin C-tagged molecule comprising a bound lanthanide ion is a troponin C fusion polypeptide, prepared as described above.

The following examples are offered to illustrate, but not to limit, the claimed invention.

### EXAMPLES

### Example 1

### Preparation of a Troponin C Binding Peptide Affinity Matrix

A troponin C binding peptide agarose (TBP-agarose) affinity matrix was prepared as follows:

Chemically-synthesized troponin C binding peptide with the sequence Ac-CGSGSSRLDYLKSSLLHLGSR-amide (SEQ ID NO:2) was resuspended at a concentration of 10 mg/ml in distilled water and then mixed with 10 volumes of a 67% slurry of Sulfolink® Coupling Gel equilibrated in 50 mM Tris, 5 mM EDTA, pH 8.5. Sulfolink® contains an iodoacetyl group that reacts with free sulfhydryls to form covalent thioether bonds. The suspension was mixed at room temperature in a sealed container for 1-10 h at 25° C. β-Mercaptoethanol was then added to a final concentration of 100 mM and incubation continued for one hour. The purpose of this step was to fully block unreacted iodoacetyl residues on the Sulfolin® gel. The TBP-agarose was then washed on a Buchner funnel with storage buffer (150 mM NaCl, 20 mM Tris HCl (pH 7.4), 1 mM EDTA, 0.05% NaN₃), and stored as a 25% slurry in storage buffer at 4° C.

### Example 2

### Expression and Affinity Purification of TnC-Jo1, TnC-LaΔC and TnC-La/SSB

This example describes the expression of troponin C-tagged Jo1 (TnC-Jo1), troponin C-tagged C-terminal truncated version of La/SSB (TnC-LaΔC), and troponin C-tagged La/SSB (TnC-La/SSB), and the affinity purification of these tagged proteins using the troponin-C binding peptide affinity matrix described in Example 1.

Cloning and Expression of TnC-LaΔC, TnC-La or TnC-Jo1

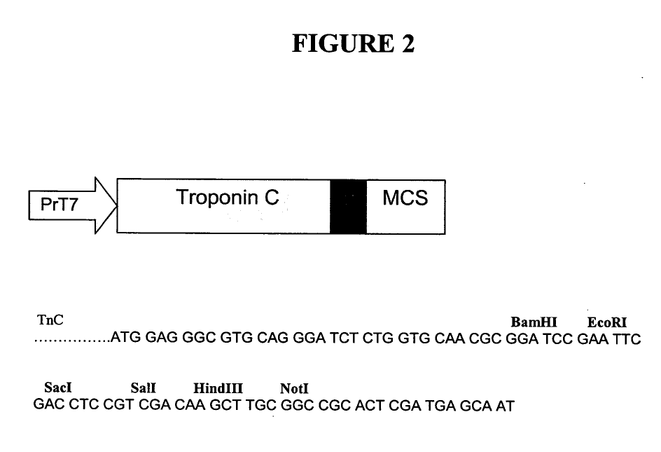

The cDNA for rabbit troponin C (rTnC) was isolated from rabbit skeletal muscle RNA using RT-PCR and the gene-specific primers rTnC-S (GCATGATCCATATGGACCAGCAGGCTGAGGCC) (SEQ ID NO:5) and rTnC-30 (CTAGCTAGGATCCCTGCACGCCCTCCATCATC) (SEQ ID NO:6). The cDNA, which was designed without its stop codon, to allow for read-through into downstream DNA fragments, was cloned into the expression vector pET22b (Novagen). The resulting TnC fusion expression vector, inducible by isopropylthioglactoside (IPTG), is depicted schematically in FIG. 2. The cDNAs for La/SSB (accession no. X13697), LaΔC (a C-terminal truncation of La/SSB prepared by PCR using forward primer 5′-CGAGCGGATCCTCCATGGCTGAAAATGGTGATAATG-3′ (SEQ ID NO:7) and reverse primer 5′-CAGACGAATTCCTAAAATTTCGTTTTCTTGCCCTG-3′ (SEQ ID NO:8)), and Jo-1 (accession no. Z11518) were isolated from human spleen cDNA (Clonetech) using gene-specific primers and RT-PCR. The cDNAs were cloned into the multiple cloning site of the TnC fusion vector using standard molecular biology techniques. The resulting fusion expression vector was transformed into BL21 (DE3) cells for expression and purification of the recombinant TnC fusion proteins.

Terrific broth (3 mL) (“Terrific Broth (Modified),” Sigma-Aldrich, catalog number T-0918) containing 100 μg/mL ampicillin and 100 μg/ml chloramphenicol was inoculated with BL21 codon plus E. coli transformed with either TnC-La/SSB, TnC-LaΔC, or TnC-Jo1. Eight hours later, the 3 mL culture was expanded to 100 mL. After another 10 hours, 100 mL of broth was added. After an additional 30 minutes, IPTG was added to a concentration of 1 mM to induce expression of the fusion protein. Cells were harvested by centrifugation after 3-4 hours and stored as a frozen paste at −20° C. or below if not used immediately.

Affinity Purification of TnC-Jo1

Frozen TnC-Jo1 E. coli (5.1 g) cells were resuspended in 100 mL of TBS (300 mM NaCl, 50 mM Tris HCl, 5 mM β-mercaptoethanol, pH 7.4) containing 1 mM EDTA. The suspension was sonicated for 2.5 minutes using a Heat-Systems W-225 sonicator and a ½ in diameter probe at 70% duty cycle and output control 8.

The suspension was centrifuged at 12,500 rpm using a Sorvall SS-34 rotor for 30 min at 4° C. The supernatant was removed and adjusted to 5 mM CaCl₂ by addition of 1 mL of 500 mM CaCl₂ per 99 mL of supernatant. A 50 mL aliquot of the supernatant (adjusted to 5 mM CaCl₂) was loaded onto a 10 mL column of TBP-agarose with an internal diameter (i.d.) of 2.5 cm, equilibrated in TBS-Ca (300 mM NaCl, 50 mM Tris HCl, 5 mM P-mercaptoethanol, 2 mM CaCl₂, pH 7.4) at a flow rate of 5 ml/min.

The column was washed at 5 mL/minute with 60 mL of TBS-Ca and eluted with 50 mL of TBS-EDTA (300 mM NaCl, 50 mM Tris HCl, 5 mM β-mercaptoethanol, 2 mM EDTA, pH 7.4). Fractions of 5 ml were collected throughout the purification.

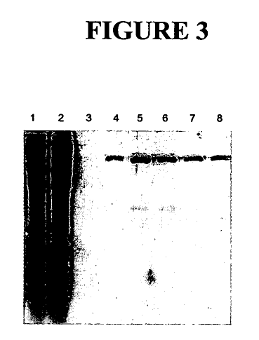

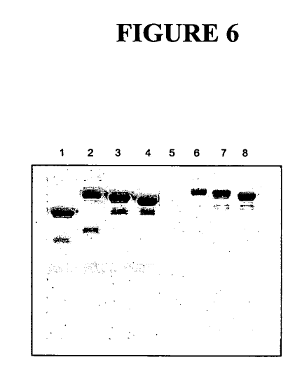

As shown in FIG. 3, purified TnC-Jo1 eluted following application of the TBS-EDTA buffer. Western blots using anti-TnC and anti-Jo-1 antibodies confirmed identity of the eluted TnC-Jo1 (FIG. 6). The lower molecular weight band observed below the TnC-Jo1 band is assumed to be a proteolytic product of TnC-Jo1, since it reacts with both anti-TnC and anti-Jo-1. The approximate molecular weight of 90,000 seen in the gel of FIG. 3 is consistent with that expected for a fusion protein of human Jo-1 (55,000 kDa) and rabbit skeletal muscle TnC (18,000). The TnC-Jo1 fusion is expressed at low levels, probably accounting for less than 1% of cellular protein. Nonetheless, the fusion protein purified quickly and with high purity.

Affinity Purification of TnC-LaΔC

Frozen TnC-LaΔC E. coli (4 g) were resuspended in 100 mL of TBS (300 mM NaCl, 50 mM Tris HCl, 5 mM β-mercaptoethanol, pH 7.4) containing 1 mM EDTA. The suspension was sonicated for 2.5 min using a Heat-Systems 2-225 sonicator and a 1/2 inch diameter probe at 70% duty cycle and output control 8.

The suspension was centrifuged at 12,500 rpm using a Sorvall SS-34 rotor for 30 min at 4° C. The supernatant was removed and adjusted to 5 mM CaCl₂ by addition of 1 mL of 500 mM CaCl₂ per 99 mL of supernatant. A 50 mL aliquot of the supernatant (adjusted to 5 mM CaCl2) was loaded onto a 10 mL column of TBP-agarose (2.5 cm i.d.), equilibrated in TBS-Ca (300 mM NaCl, 50 mM Tris HCl, 5 mM β-mercaptoethanol, 2 mM CaCl₂, pH 7.4).

The column was washed at 5 mL/minute with 60 mL of TBS-Ca and column was eluted with 50 mL of TBS-EDTA (300 mM NaCl, 50 mM Tris HCl, 5 mM α-mercaptoethanol, 2 mM EDTA, pH 7.4). Fractions of 5 mL were collected throughout the purification.

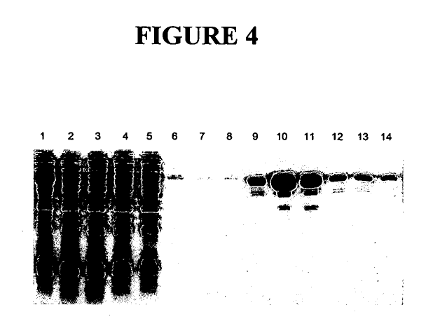

As shown in FIG. 4, purified TnC-LaΔC eluted following application of the TBS-EDTA buffer. Western blots using anti-TnC and anti-La/SSB antibodies confirmed the identity of the eluted TnC-LaΔC (FIG. 6). The approximate molecular weight observed in the gel (˜60,000) was consistent with that expected for a fusion protein of truncated La/SSB (44,000) and rabbit skeletal muscle troponin C (18,000).

Affinity Purification of TnC-La/SSB

Frozen TnC-La/SSB E. coli (5.7 g) were resuspended in 100 mL of TBS (300 mM NaCl, 50 mM Tris HCl, 5 mM β-mercaptoethanol, pH 7.4) containing 1 mM EDTA. The suspension was sonicated for 2.5 min using a Heat-Systems 2-225 sonicator and a 1/2 inch diameter probe at 70% duty cycle and output control 8 or equivalent).

The suspension was centrifuged at 12, 500 rpm using a Sorvall SS-34 rotor for 30 min at 4° C. The supernatant was removed and adjusted to 5 mM CaCl₂ by addition of 1 mL of 500 mM CaCl₂ per 99 mL of supernatant. A 50 mL aliquot of the supernatant (adjusted to 5 mM CaCl₂) was loaded onto a 10 mL column of TBP agarose (2.5 cm i.d.), equilibrated in TBS-Ca (300 mM NaCl, 50 mM Tris HCl, 5 mM β-mercaptoethanol, 2 mM CaCl₂, pH 7.4).

The column was washed at 5 mL/minute with 60 mL of TBS-Ca and the column was eluted with 50 mL of TBS-EDTA (300 mM NaCl, 50 mM Tris HCl, 5 mM β-mercaptoethanol, 2 mM EDTA, pH 7.4). Fractions of 5 mL were collected throughout the purification.

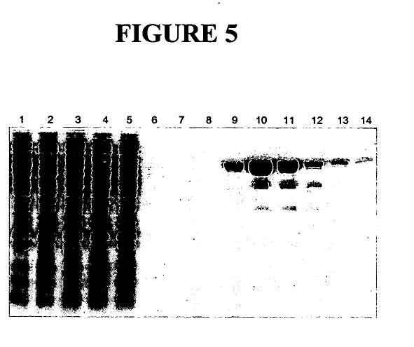

As shown in FIG. 5, purified TnC-La/SSB eluted following application of the TBS-EDTA buffer. Western blots using anti-TnC and anti-La/SSB antibodies confirmed the identity of the eluted TnC-LaΔC (FIG. 6). The approximate molecular weight observed in the gel (˜73,000) was consistent with that expected for a fusion protein of full length La/SSB (55,000) and rabbit skeletal muscle troponin C.

Characterization of TnC Fusion Proteins by Western Blot

FIG. 6 shows a Coomassie blue stained SDS-PAGE gel and a corresponding Western blot of TBP-La/SSB (control), TnC-Jo1, TnC-LaΔC, and TnC-La/SSB. The western blot was developed using a 1:100 dilution of a chicken anti-rabbit troponin C as primary antibody (derived using native rabbit skeletal muscle troponin C as immunogen) and a 1:2000 dilution of rabbit anti-chicken IgG as secondary antibody. The blot was developed using the NBT/BCIP substrate system. Only the TnC fusion proteins reacted with the chicken anti rabbit troponin C, thereby confirming the presence of the troponin C fusion tag.

### Example 3

### Preparation of a Troponin C Affinity Matrix

A troponin C agarose (TnC-agarose) affinity matrix was prepared as follows:

Rabbit skeletal muscle troponin C (140 mg) in 70 ml of 500 mM NaCl, 1 mM sodium phosphate (pH 7.0), 0.05% NaN₃, was mixed with 80 g (wet weight) of 4% crosslinked Glyoxal Agarose (ABT Beads, Lowell, Mass.) that had been washed under vacuum on a Buchner funnel with 200 mM sodium phosphate (pH 6.4). Ten milliliters of 200 mM sodium phosphate (pH 6.4) was added and the pH of the suspension was adjusted to pH 6.4 (if needed). The suspension was stirred at room temperature for 20 minutes after which 10 ml of 2 M sodium cyanoborohydride was added. The suspension was gently stirred for 2.5 hours at room temperature to facilitate coupling of the troponin C. Unreacted glyoxal groups were then blocked by addition of 1.0 ml of ethanolamine, followed by stirring at room temperature for an additional 30 min. The gel was then washed extensively with 10 mM TrisCl (pH 7.4), 150 mM NaCl, 1 mM EDTA, 0.05% NaN₃ and stored at 4° C.

### Example 4

### Expression and Affinity Purification of TBP-LaΔC

This example describes the expression of troponin C binding peptide-tagged LaΔC and the affinity purification of this tagged protein using the TnC affinity matrix prepared as described in Example 3.

Cloning and Expression of TBP-LaΔC

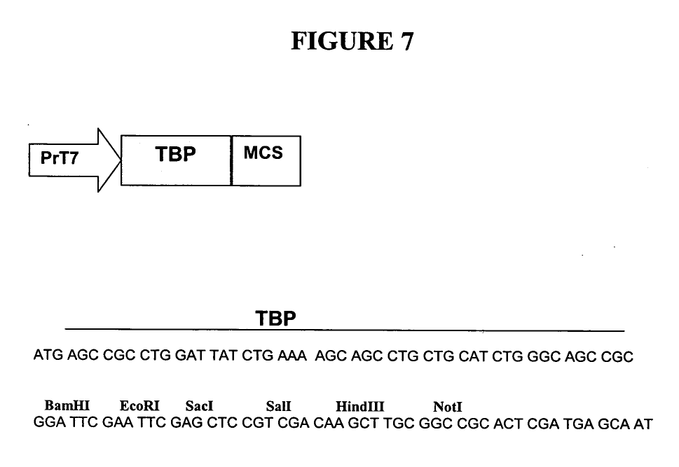

An oligonucleotide coding for the peptide sequence SRLDYLKSSLLHLGSR (SEQ ID NO:1) was used in the design of an expression vector for producing troponin C binding peptide (TBP) fusion proteins for affinity purification on a troponin C affinity matrix. Construction of the vector was facilitated by the simultaneous PCR amplification of the E. coli LacZ gene using the oligonucleotides Z3 (CATATGAGCCGCCTGGATTATCTGAAAAGCAGCCTGCTGCATCTGGG CAGCCGCGGATCCGTCGTTTTACAACGTCGTG) (SEQ ID NO:9) and Z2 (CAGAGAATTCGAAGGATCCCGGCTTTATTATTTTTGACACCAG) (SEQ ID NO:10). The resulting TBP-LacZ DNA fragment was cloned into pET22b as a NdeI-EcoRI fragment. The TBP fusion vector was obtained by digesting the pET22b-TBP-LacZ vector with BamHI, which resulted in the removal of the lacZ gene. The resulting expression vector is shown schematically in FIG. 7. The cDNA for a C-terminal truncated La/SSB (LaΔC; accession #X13697) was isolated from human spleen cDNA (Clonetech) using gene-specific primers and RT-PCR. The cDNA was cloned into the TBP fusion vector using standard molecular biology techniques. The resulting recombinant plasmid was transformed into BL21 (DE3) cells for expression and purification of the recombinant protein.

Terrific broth (100 mL) containing 100 μg/mL ampicillin and 100 μg/ml chloramphenicol was inoculated with BL21 codon plus E. coli transformed with TBP-LaΔC. Eleven hours later, the culture was expanded to 900 mL. After 7.5 hours, 900 mL of broth was added. After an additional 30 minutes, isopropylthioglactoside was added to a concentration of 1 mM to induce expression of the fusion protein. Cells were harvested by centrifugation after 4.5 hours.

Affinity Purification of TBP-LaΔC

Frozen TBP-LaΔC E. coli (4.3 g) were resuspended in 100 mL of TBS (900 mM NaCl, 50 mM Tris HCl, 5 mM P-mercaptoethanol, pH 7.4). The suspension was sonicated for 2.5 min using a Heat-Systems 2-225 sonicator and a 1/2 inch diameter probe at 70% duty cycle and output control 8.

The suspension was centrifuged at 12,500 rpm using a Sorvall SS-34 rotor for 30 min at 4° C. The supernatant was removed and adjusted to 5 mM CaCl₂ by addition of 1 mL of 500 mM CaCl₂ per 99 mL of supernatant. A 45 mL aliquot of the supernatant (adjusted to 5 mM CaCl₂) was loaded onto a 20 mL column of TnC-agarose (2.5 cm i.d.), equilibrated in TBS-Ca (900 mM NaCl, 50 mM Tris HCl, 5 mM P-mercaptoethanol, 2 mM CaCl₂, pH 7.4).

The column was washed at 5 mL/minute with 80 mL of TBS-Ca and eluted with 60 mL of TBS-EDTA (900 mM NaCl, 50 mM Tris HCl, 5 mM β-mercaptoethanol, 2 mM EDTA, pH 7.4). Fractions of 10 ml were collected throughout the load and wash stages to 130 ml, and fractions of 10 ml were collected thereafter.

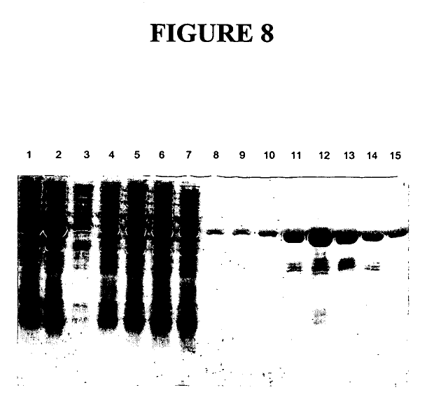

As shown in FIG. 8, purified TBP-LaΔC eluted following application of the TBS-EDTA buffer. Western blots using anti-TBP and anti-La/SSB antibodies confirmed the identity of the eluted TBP-LaΔC. The approximate molecular weight observed in the gel (˜57,000) was consistent with that expected for a fusion protein of truncated La/SSB (55,000) and the TBP peptide (2,200).

### Example 5

### Preparation of Troponin I Affinity Matrix

A troponin I affinity matrix can be used to purify troponin C-tagged moieties (e.g. Tn-C fusion proteins) under denaturing conditions. A troponin I affinity matrix (TnI-agarose) was prepared as follows:

The cDNA for human cardiac troponin-I was isolated from human heart cDNA (Clonetech) using gene-specific primers and RT-PCR. To facilitate coupling to the affinity support material via the N-terminus, a recombinant mutated version was prepared. This version of human cardiac troponin-I, referred to as N-3cys-8ser-TnI, consisted of full length human cardiac troponin-I (accession no. CAA62301) in which the two internal cysteines at positions 80 and 97 were mutated to serine residues and in which the sequence N′-CCCSSSSSSSS-C′ (SEQ ID NO:3) was added to the N-terminus of the protein. The cDNA for N-3cys-8ser-TnI was cloned into the a pET22B expression vector using standard molecular biology techniques. The N-3cys-8ser-TnI was subsequently expressed in E. coli and purified in the presence of 8M urea by calcium dependent affinity chromatography on rabbit skeletal muscle troponin-C immobilized on Sepharose.

Purified N-3cys-8ser-TnI (˜60 mg), in a buffer comprised of 8M urea, 50 mM Tris HCl, 100 mM NaCl, 4 mM EDTA (pH 7.4)) at a concentration of approximately 1.5 mg/ml was mixed with Sulfolin® Coupling Gel (10 ml settled volume) equilibrated in 50 mM Tris, 5 mM EDTA, pH 8.5. The suspension was mixed at room temperature in a sealed container for 1 h at 25° C. β-Mercaptoethanol was then added to a final concentration of 100 mM and incubation continued for one hour to fully block unreacted iodoacetyl residues on the Sulfolink® gel. The TnI-agarose was then poured into a 2.5 cm i.d. column, equilibrated in 8M urea, 50 mM Tris HCl, 100 mM NaCl, 4 mM EDTA (pH 7.4)) and stored at 4° C. until used.

### Example 6

### Expression and Affinity Purification of TnC-Ro52

This example describes the expression of troponin C-tagged Ro52 and purification on the troponin I affinity matrix described in Example 5, under denaturing conditions.

Cloning and Expression of TnC-Ro52

The cDNA for human Ro52 (Accession no. NM_{—}003141) was isolated from human spleen cDNA (Clonetech) using gene-specific primers and RT-PCR. The cDNA was cloned into the TnC fusion vector described in Example 2, using standard molecular biology techniques. The resulting recombinant fusion expression vector was transformed into BL21 (DE3) cells.

Terrific broth (3 mL) containing 100 μg/mL ampicillin and 100 μg/ml chloramphenicol was inoculated with BL21 codon plus E. coli transformed with TnC-Ro52. Eight hours later, the 3 mL culture was expanded to 100 mL. After 10 hours, 150 mL of cold (4° C.) broth was added. After an additional 75 minutes, isopropylthioglactoside was added to a concentration of 1 mM to induce expression of the fusion protein. Cells were harvested by centrifugation after 4 hours and stored as a frozen paste at −20° C. or below if not used immediately.

Affinity Purification of TnC-Ro52

Frozen TnC-Ro52 E. coli (3.8 g) cells were resuspended in 100 mL of 150 mM NaCl, 50 mM Tris HCl, 5 mM β-mercaptoethanol (pH7.4). The suspension was sonicated for 2.5 minutes using a Heat-Systems W-225 sonicator and a ½ in diameter probe at 70% duty cycle and output control 8.

The suspension was centrifuged at 12,500 rpm using a Sorvall SS-34 rotor for 30 min at 4° C. The supernatant was discarded and the pellets that contained the insoluble TnC-Ro52 fusion protein were resuspended in 100 ml of 8M urea, 50 mM Tris HCl, 100 mM NaCl (pH 7.4). The suspension was sonicated for 2.5 minutes using a Heat-Systems W-225 sonicator and a ½ in diameter probe at 70% duty cycle and output control 8 and centrifuged at 12,500 rpm using a Sorvall SS-34 rotor for 30 min at 4° C.

The supernatant that contained the solubilized TnC-Ro52 fusion protein was adjusted to 10 mM CaCl₂, 300 mM NaCl. A 20 mL aliquot of the supernatant was loaded onto a 10 mL column of TnI-agarose (2.5 cm i.d.), equilibrated in UBS-Ca (8M urea, 50 mM Tris HCl, 300 mM NaCl, 4 mM CaCl₂, 10 mM β-mercaptoethanol (pH 7.4)) at a flow rate of 0.5 ml/min.

The column was washed at 0.5-1.0 mL/minute with 40 mL of UBS-Ca and eluted with 50 mL of UBS-EDTA (8M urea, 50 mM Tris HCl, 300 mM NaCl, 4 mM EDTA, 10 mM β-mercaptoethanol (pH 7.4)). Fractions of 5 ml were collected throughout the purification.

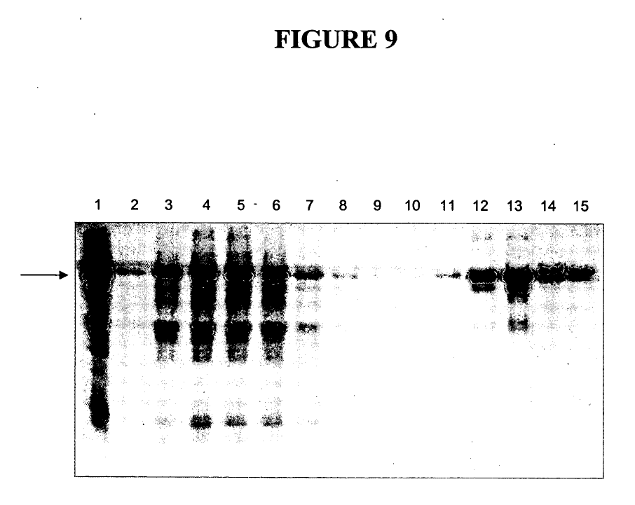

As shown in FIG. 9, purified TnC-Ro52 eluted following application of the UBS-EDTA buffer. The approximate molecular weight of 70 kDa seen in the gel of FIG. 9 is consistent with that expected for a fusion protein of human Ro52 (52,000 kDa) and rabbit skeletal muscle TnC (18,000).

### Example 7

### Comparison of the Purification of His-Tagged La Antigen and TnC-Tagged La Antigen

To illustrate the advantages of the TnC fusion system for purification of recombinant proteins, side by side purifications were performed using a polyhistidine-tagged La antigen (His-La) and a rabbit skeletal muscle troponin C-tagged C-terminal truncated La antigen (TnC-LaΔC).

Cloning and Expression of His-La

The cDNA for La/SSB (accession no. X13697) was isolated from human spleen cDNA (Clonetech) using gene-specific primers and RT-PCR. The cDNA was cloned into the pTrchisA fusion vector using standard molecular biology techniques. The resulting recombinant plasmid was transformed into BL21 (DE3) cells for expression and purification of the recombinant protein. The resulting expressed protein consisted of a C-terminal His6-tagged La/SSB fusion protein.

Terrific broth (3 mL) containing 100 μg/mL ampicillin was inoculated with BL21 DE3 E. coli transformed with His-La. The culture was incubated in a shaking incubator at 37° C. Ten hours later, 0.5 ml of the culture was used to inoculate 80 mL of fresh broth. After 10 hours, 160 mL of room temperature broth was added. After an additional 15 minutes, IPTG was added to a concentration of 1 mM to induce expression of the fusion protein. Cells were harvested by centrifugation after 4 hours and stored as a frozen paste at −20° C. or below if not used immediately.

Affinity Purification of His-La

Frozen His-La E. coli cells (4.0 g) were resuspended in 100 mL of 50 mM TrisCl, 300 mM NaCl (pH 7.4). The suspension was sonicated for 2.5 minutes using a Heat-Systems W-225 sonicator and a ½ in diameter probe at 70% duty cycle and output control 8. After centrifugation for 20 minutes at 12,500 rpm in a Sorvall SS-34 rotor, the supernatant was adjusted to 50 mM Tris HCl, 300 mM NaCl, 30 mM imidazole (pH 7.4) and 30 ml was loaded at 5 ml/min onto a 10 ml column of nickel chelated iminodiacetic acid agarose (2.5 cm, i.d.) equilibrated in 50 mM Tris HCl, 300 mM NaCl, 30 mM imidazole (pH 7.4). The column was washed with 60 ml of column equilibration buffer and the His-La fusion protein was eluted with 60 ml of 50 mM Tris HCl, 300 mM NaCl, 300 mM imidazole (pH 7.4). Fractions of 10 ml were collected during the load and wash steps and fractions of 5 ml were collected during the elution with 300 mM imidazole.

Cloning and Expression of TnC-LaΔC

The cDNA for a C-terminal truncated La/SSB (accession #X13697) was isolated from human spleen cDNA (Clonetech) using gene-specific primers and RT-PCR. The cDNA was cloned into the rabbit skeletal muscle TnC fusion vector using as described in Example 2, using standard molecular biology techniques. The resulting recombinant plasmid was transformed into BL21 codon plus cells for expression and purification of the recombinant protein. The resulting expressed protein consisted of an N-terminal rabbit skeletal muscle TnC-tagged C-terminal truncated La/SSB fusion protein (TnC-LaΔC).

Terrific broth (3 mL) containing 100 μg/mL ampicillin and 34 mg/ml chloramphenicol was inoculated with BL21 codon plus E. coli transformed with TnC-LaΔC. The culture was incubated in a shaking incubator at 37° C. Ten hours later, 0.5 ml of the culture was used to inoculate 80 mL of fresh broth. After 10 hours, 160 mL of room temperature broth was added. After an additional 15 minutes, isopropylthioglactoside was added to a concentration of 1 mM to induce expression of the fusion protein. Cells were harvested by centrifugation after 4 hours and stored as a frozen paste at −20° C. or below if not used immediately.

Affinity Purification of TnC-LaΔC

Frozen TnC-LaΔC E. coli cells (4.4 g) were resuspended in 100 mL of 50 mM TrisCl pH 7.4, 300 mM NaCl (pH 7.4). The suspension was sonicated for 2.5 minutes using a Heat-Systems W-225 sonicator and a ½ in diameter probe at 70% duty cycle and output control 8. After centrifugation for 20 minutes at 12,500 rpm in a Sorvall SS-34 rotor, the supernatant was saved and adjusted to 50 mM Tris HCl, 300 mM NaCl, 10 mM CaCl₂ (pH 7.4) and 30 ml was loaded at 5 ml/min onto a 10 ml column of TBP-agarose (2.5 cm, i.d.) equilibrated in 50 mM Tris HCl, 300 mM NaCl, 4 mM CaCl₂ (pH 7.4). The column was washed with 60 ml of column equilibration buffer and the TnC-LaΔC fusion protein was eluted with 60 ml of 50 mM Tris HCl, 300 mM NaCl, 4 mM EDTA (pH 7.4). Fractions of 10 ml were collected during the load and wash steps and fractions of 5 ml were collected during the elution with buffer containing EDTA.

Comparison of Affinity Purification of His-La and TnC-LaΔC

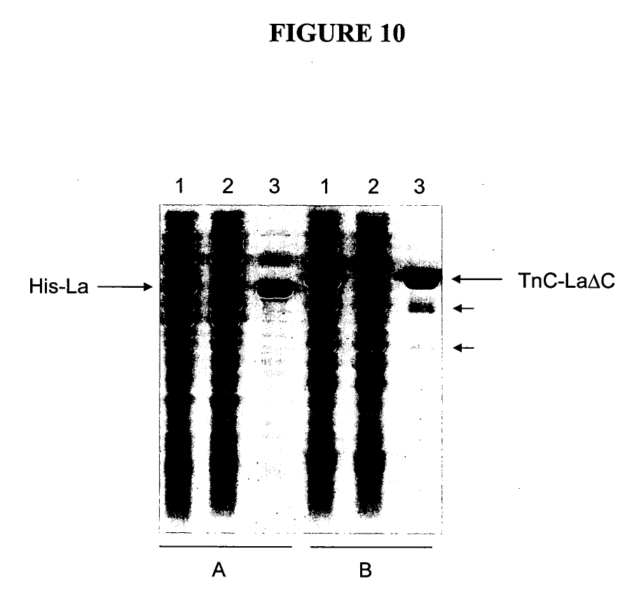

A comparison of His-La and TnC-LaΔC purifications is shown in FIG. 10. FIG. 10 shows SDS-PAGE profiles of the respective column loads (lanes 1), flow-through fractions (lanes 2) and peak eluted fraction for the recombinant fusion proteins (lanes 3) for His-La (A) and TnC-LaΔC (B) respectively. The His-La fusion protein eluted with approximately 80% purity and was contaminated with numerous proteins of higher and lower molecular weight. In contrast, the TnC-LaΔC fusion eluted with a purity of ˜95%. Few, if any, contaminants were apparent. The minor lower molecular weight bands reacted with anti-rabbit skeletal muscle TnC antibodies in western blot, indicating that they likely represent proteolytic breakdown or incomplete translation products of the TnC-LaΔC fusion.

### Example 8

### Proteolytic Cleavage of a Troponin C-Tagged Fusion Protein, TnC-LaΔC, Using an Internal Thrombin Recognition Site

This example describes the proteolytic cleavage of a troponin C tag from a troponin C-tagged protein, using an internal protease recognition site.

LaΔC was cloned into a troponin C expression vector containing a thrombin recognition site between sequences encoding a troponin C tag and a multiple cloning site. Thrombin cleavage was performed on purified TnC-LaΔC using a Thrombin Cleavage Capture Kit (Novagen, Madison, Wis.), according to the manufacturer's directions. Briefly, 10 μg of purified TnC-LaΔC was mixed with 0.0125 to 0.1 units of biotinylated thrombin in a final volume of 50 μl and incubated at room temperature. After 48 hours, the reaction was quenched by addition of 16.7 μl of 4× Laemmli sample buffer, and 10 μl aliquots were electrophoresed on a 12.5% SDS-PAGE mini gel.

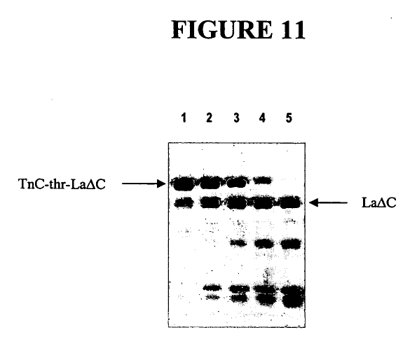

The results of the thrombin proteolytic cleavage are shown in FIG. 11. The gel demonstrates proteolytic cleavage of the TnC tag, with apparent complete cleavage when 0.1 units of thrombin was used, resulting in separation of the troponin C tag from LaΔC.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entireties for all purposes.

1. A method of purifying a troponin-tagged molecule, said method comprising contacting the troponin-tagged molecule with an affinity matrix that comprises a cognate ligand of the troponin tag, thereby immobilizing the troponin-tagged molecule on the affinity matrix.

2. A method according to claim 1, wherein said troponin-tagged molecule is contacted with said affinity matrix in the presence of calcium.

3. A method according to claim 2, wherein the affinity of binding between the troponin tag and said cognate ligand comprises a K_{d} greater than 10 nM.

4. A method according to claim 2, wherein said troponin-tagged molecule is released from said affinity matrix by adding an agent that chelates calcium.

5. A method according to claim 4, wherein said agent that chelates calcium is selected from the group consisting of EDTA, EGTA, BAPTA, citrate, and phosphate.

6. A method according to claim 1, wherein said troponin-tagged molecule is a fusion protein that comprises a troponin molecule and a polypeptide that is not a troponin molecule.

7. A method according to claim 6, wherein said fusion protein is produced recombinantly.

8. A method according to claim 6, wherein said fusion protein comprises at least one troponin molecule at the N-terminus.

9. A method according to claim 6, wherein said fusion protein comprises at least one troponin molecule at the C-terminus.

10. A method according to claim 6, wherein said fusion protein comprises at least one troponin molecule at an amino acid residue between the N-terminus and the C-terminus.

11. A method according to claim 6, wherein said fusion protein comprises a linker between said troponin molecule and said polypeptide that is not a troponin molecule.

12. A method according to claim 11, wherein said linker is a polypeptide.

13. A method according to claim 12, wherein said polypeptide linker comprises a protease recognition site.

14. A method according to claim 13, wherein said polypeptide that is not a troponin molecule is released from said affinity matrix by protease cleavage at the protease recognition site.

15. A method according to claim 1, wherein said troponin-tagged molecule comprises at least one molecule of troponin C, or a fragment or analogue thereof that is capable of specifically binding said cognate ligand on said affinity matrix.

16. A method according to claim 15, wherein said affinity matrix comprises a troponin C binding peptide.

17. A method according to claim 16, wherein said troponin C binding peptide comprises the sequence SRLDYLKSSLLHLGSR (SEQ ID NO:1), or a fragment or analogue thereof that is capable of specifically binding troponin C.

18. A method according to claim 17, wherein said troponin C binding peptide further comprises a cysteine residue at the N-terminus, and said affinity matrix is formed by reacting the troponin C binding peptide with a thiol reactive matrix.

19. A method according to claim 16, wherein the affinity matrix comprises a substrate selected from the group consisting of cross-linked polysaccharide, agarose, ceramic, metal, glass, plastic, and cellulose.

20. A method according to claim 16, wherein said troponin C-tagged molecule is released from said affinity matrix by adding a releasing agent, and wherein said releasing agent comprises a troponin C binding peptide.

21. A method according to claim 15, wherein said affinity matrix comprises troponin I, or a fragment or analogue thereof that is capable of specifically binding troponin C.

22. A method according to claim 21, wherein said troponin I is mutated to eliminate internal cysteine residues or to replace internal cysteine residues with other amino acids, wherein said troponin I comprises the sequence CCCSSSSSSSS (SEQ ID NO:3) at the N-terminus or the sequence SSSSSSSSCCC (SEQ ID NO:4) at the C-terminus, and wherein said affinity matrix is formed by reacting said troponin I with a thiol reactive matrix.

23. A method according to claim 22, wherein said internal cysteine residues are replaced with amino acids selected from the group consisting of serine and threonine.

24. A method according to claim 21, wherein the affinity matrix comprises a substrate selected from the group consisting of cross-linked polysaccharide, agarose, ceramic, metal, glass, plastic, and cellulose.

25. A method according to claim 21, wherein said troponin C-tagged molecule is released from said affinity matrix by adding a releasing agent, and wherein said releasing agent comprises troponin I, or a fragment or analogue thereof that is capable of specifically binding troponin C.

26. A method according to claim 21, wherein at least part of said method is performed in the presence of a denaturing agent.

27. A method according to claim 26, wherein said denaturing agent comprises urea.

28. A method according to claim 1, wherein said affinity matrix comprises troponin C, or a fragment or analogue thereof that is capable of binding the troponin tag on said troponin-tagged molecule.

29. A method according to claim 28, wherein said troponin-tagged molecule comprises at least one molecule of a troponin C binding peptide.

30. A method according to claim 29, wherein said troponin C binding peptide comprises the sequence SRLDYLKSSLLHLGSR (SEQ ID NO:1), or a fragment or analogue thereof that is capable of specifically binding troponin C.

31. A method according to claim 28, wherein said troponin C binding peptide-tagged molecule is released from said affinity matrix by adding a releasing agent, and wherein said releasing agent comprises troponin C, or a fragment or analogue thereof that is capable of specifically binding said troponin C binding peptide.

32. A method according to claim 28, wherein said troponin-tagged molecule comprises at least one molecule of troponin I, or a fragment or analogue thereof that is capable of specifically binding troponin C.

33. A method according to claim 32, wherein said troponin I-tagged molecule is released from said affinity matrix by adding a releasing agent, and wherein said releasing agent comprises troponin C, or a fragment or analogue thereof that is capable of specifically binding said troponin I.

34. A method according to claim 32, wherein at least part of said method is performed in the presence of a denaturing agent.

35. A method according to claim 34, wherein said denaturing agent comprises urea.

36. A method according to claim 28, wherein said affinity matrix is formed by reacting troponin C with a cyanogen bromide or glyoxal activated matrix.

37. A method according to claim 28, wherein said affinity matrix is formed by irreversibly linking troponin C to a substrate via reactive amino groups of said troponin C.

38. A method according to claim 28, wherein the affinity matrix comprises a substrate selected from the group consisting of cross-linked polysaccharide, agarose, ceramic, metal, glass, plastic, and cellulose.

39. A molecule purified according to the method of claim 1.

40. A molecule according to claim 39, wherein said molecule is a troponin-tagged fusion protein.

41. A method for detecting a troponin-tagged molecule according to claim 39, comprising contacting said troponin-tagged molecule with an antibody that specifically recognizes the troponin tag.

42. An affinity matrix comprising troponin C, or a fragment or analogue thereof that is capable of specifically binding to a troponin C binding peptide or troponin I, wherein said troponin C is attached to a substrate.

43. An affinity matrix according to claim 42, further comprising a bound troponin C binding peptide- or troponin I-tagged molecule.

44. A method of producing an affinity matrix according to claim 42, comprising reacting said troponin C with a cyanogen bromide or glyoxal activated matrix.

45. A method of producing an affinity matrix according to claim 42, comprising irreversibly linking said troponin C to a substrate via reactive amino groups of said troponin C.

46. An affinity matrix comprising a troponin C binding peptide attached to a substrate.

47. An affinity matrix according to claim 46, wherein said troponin C binding peptide comprises the sequence SRLDYLKSSLLHLGSR (SEQ ID NO:1), or a fragment or analogue thereof that is capable of specifically binding to troponin C.

48. An affinity matrix according to claim 46, further comprising a bound troponin C-tagged molecule.

49. A method of producing an affinity matrix according to claim 47, wherein said troponin C binding peptide further comprises a cysteine residue at the N-terminus, and said method comprises reacting the troponin C binding peptide with a thiol reactive matrix.

50. An affinity matrix comprising troponin I, or a fragment or analogue thereof that is capable of specifically binding to troponin C, wherein said troponin I is attached to a substrate.

51. An affinity matrix according to claim 50, further comprising a bound troponin C-tagged molecule.

52. A method of producing an affinity matrix according to claim 50, wherein said troponin I is mutated to eliminate internal cysteine residues or to replace internal cysteine residues with other amino acids, wherein said troponin I comprises the sequence CCCSSSSSSSS (SEQ ID NO:3) at the N-terminus or the sequence SSSSSSSSCCC (SEQ ID NO:4) at the C-terminus, and said method comprises reacting said troponin I with a thiol reactive matrix.

53. A method according to claim 52, wherein said internal cysteine residues are replaced with amino acids selected from the group consisting of serine and threonine.

54. A kit comprising an affinity matrix according to claim 42 and instructions for use in a method of purifying a troponin C binding peptide-tagged molecule.

55. A kit according to claim 54, further comprising components for producing a troponin C binding peptide-tagged molecule.

56. A kit comprising an affinity matrix according to claim 42 and instructions for use in a method of purifying a troponin I-tagged molecule.

57. A kit according to claim 56, further comprising components for producing a troponin I-tagged molecule.

58. A kit comprising an affinity matrix according to claim 46 and instructions for use in a method of purifying a troponin C-tagged molecule.

59. A kit according to claim 58, further comprising components for producing a troponin C-tagged molecule.

60. A kit comprising an affinity matrix according to claim 50 and instructions for use in a method of purifying a troponin C-tagged molecule.

61. A kit according to claim 60, further comprising components for producing a troponin C-tagged molecule.

62. A method for detecting a troponin C-tagged molecule, said method comprising contacting a troponin C-tagged molecule with lanthanide ions.

63. A method according to claim 62, wherein said lanthanide ions are selected from the group consisting of lanthanum, terbium, europium, and gadolinium.

64. A method according to claim 62, wherein said troponin C-tagged molecule is detected by luminescence of lanthanide ions bound to the troponin C-tagged molecule.

65. A method according to claim 62, wherein said troponin C-tagged molecule is a troponin C fusion protein.

66. A method according to claim 64, wherein said method is used in an application selected from the group consisting of high throughput screening, study of receptor-ligand interaction, and study of binding kinetics.


SEQUENCE LISTING (12 sequences)

SEQ ID NO: 1 - protein, 16 aa, Artificial Sequence
  Synthetic Construct
SRLDYLKSSLLHLGSR

SEQ ID NO: 2 - protein, 21 aa, Artificial Sequence
  Synthetic Construct
CGSGSSRLDYLKSSLLHLGSR

SEQ ID NO: 3 - protein, 11 aa, Artificial Sequence
  Synthetic Construct
CCCSSSSSSSS

SEQ ID NO: 4 - protein, 11 aa, Artificial Sequence
  Synthetic Construct
SSSSSSSSCCC

SEQ ID NO: 5 - DNA, 32 bp, Artificial Sequence
  Synthetic Construct
gcatgatccatatggaccagcaggctgaggcc

SEQ ID NO: 6 - DNA, 32 bp, Artificial Sequence
  Synthetic Construct
ctagctaggatccctgcacgccctccatcatc

SEQ ID NO: 7 - DNA, 36 bp, Artificial Sequence
  Synthetic Construct
cgagcggatcctccatggctgaaaatggtgataatg

SEQ ID NO: 8 - DNA, 35 bp, Artificial Sequence
  Synthetic Construct
cagacgaattcctaaaatttcgttttcttgccctg

SEQ ID NO: 9 - DNA, 79 bp, Artificial Sequence
  Synthetic Construct
catatgagccgcctggattatctgaaaagcagcctgctgcatctgggcagccgcggatccgtcgttttacaacgtcgtg

SEQ ID NO: 10 - DNA, 43 bp, Artificial Sequence
  Synthetic Construct
cagagaattcgaaggatcccggctttattatttttgacaccag

SEQ ID NO: 11 - DNA, 86 bp, Artificial Sequence
  Synthetic Construct
atggagggcgtgcagggatctctggtgcaacgcggatccgaattcgacctccgtcgacaagcttgcggccgcactcgatgagcaat

SEQ ID NO: 12 - DNA, 104 bp, Artificial Sequence
  Synthetic Construct
atgagccgcctggattatctgaaaagcagcctgctgcatctgggcagccgcggattcgaattcgagctccgtcgacaagcttgcggccgcactcgatgagcaat